(19) United States
(12) Patent Application Publication
(10) Pub. No.: US 2012/0238740 A1
(43) Pub. Date: Sep. 20, 2012

(54) NON-POLAR AND POLAR LEAVING GROUPS

(75) Inventors: Keith Graham, Berlin (DE); Mathias Berndt, Berlin (DE); Dae Yoon Chi, Yongsan-Gu (KR); Byoung Se Lee, Nam-Gu (KR); Sandip S. Shinde, Maharashtra (IN); Hee Seup Kil, Goyang-Si (KR); Sang Ju Lee, Seongdong-Gu (KR); Jin-Sook Ryu, Kangnam-Gu (KR); Seung Jun Oh, Yeongdeungpo-Gu (KR)

(73) Assignee: BAYER PHARMA AKTIENGESELLSCHAFT, Berlin (DE)

(21) Appl. No.: 13/383,231

(22) PCT Filed: Jul. 6, 2010
(86) PCT No.: PCT/EP10/04111

§ 371 Date: Jun. 5, 2012

(30) Foreign Application Priority Data
Jul. 11, 2009 (EP) 09075294.0

(51) Int. Cl. C07H 1/00; C07C 309/31; C07J 31/00; C07C 309/73; C07C 303/28; C07H 19/06; C07C 253/30; C07C 253/34; C07D 405/12; C07J 43/00; C07C 309/66; C07C 309/65; C07C 227/16; C07C 227/40; C07H 1/06; C07D 213/64; C07D 237/16; C07D 451/02; B01D 3/34; C07C 309/30

(52) U.S. Cl. 536/28.2; 562/45; 552/540; 558/48; 536/28.54; 558/401; 548/311.1; 540/108; 558/51; 560/39; 546/291; 540/113; 544/240; 546/132; 203/28

(57) ABSTRACT

The present invention provides novel and advantageous processes for preparing and purifying pharmaceuticals The processes comprise a nucleophilic reaction wherein a modified leaving group L^{M}, which has increased lipophilicity, of a vector in a nucleophilic reaction which offers a convenient and time-saving way to purify the product from non-reacted precursors vector-L^{M} and by-products L^{M}.

### FIELD OF THE INVENTION

The invention generally relates to the preparation of pharmaceuticals. In particular, this invention relates to processes and kits for carrying out an efficient “liquid phase” nucleophilic substitution reaction with a nucleophilic reagent X on a precursor targeting vector comprising leaving group L^{M} to the targeting vector, whereby the leaving group L^{M} has increased lipophilicity. The methods and kits of the present invention allow a simple purification of the desired pharmaceutical vector-X from non-reacted precursors and by-products still containing said leaving group L_{M}.

### BACKGROUND OF THE INVENTION

In the preparation of many pharmaceuticals, including radiohalogenated pharmaceuticals, nucleophilic substitution reactions as depicted in Scheme 1a are useful and commonly employed.

X+vector-L→vector-X+L Scheme 1a:
wherein vector is a targeting vector,
X is a nucleophilic reagent and
L is a leaving group.

For example, U.S. Pat. No. 5,565,185 discloses a non-carrier process of radiolabelling meta-iodobenzylguanidine (MIBG) by halodestannylation. However, the process is disadvantageous in that a number of impurities remain in solution with the radiolabeled MIBG. In particular, toxic tin by-products remain in solution and must be separated before the radiolabeled MIBG is ready for use.

Strategies to remove by-products, such as excess precursors had to be established for successful (radio-) synthesis and subsequent safe administration of compounds of clinical interest. Such reactions often employ non-radioactive organic precursors in amounts that are in large excess relative to the amount of the radiolabelling agent used. Excess precursors must then be removed from the reaction mixture before the radiolabeled compound can be applied to a patient for diagnostic and/or therapeutic applications.

In the case of radiohalogen pharmaceuticals, X can generally be easily separated from the other species in the reaction mixture using for example alumina solid phase extraction. Moreover, those of skill in the art are generally aware of methods to remove other radiolabeled, nucleophilic species such as ¹¹C-compounds or nucleophilic compounds in general, using standard purification protocols.

It is, however, generally more difficult to separate vector-X and vector-L. In many cases, it is particularly important to separate vector-X from unlabeled targeting vector vector-L, because vector-L can compete with and therefore interfere with binding of vector-X to its target. If this competition occurs, this effect may lead to sub-optimal performance characteristics of the radiopharmaceutical. This is particularly the case for receptor-binding (i.e. specific targeting) radiopharmaceuticals.

The purification of vector-X from vector-L is commonly accomplished by employing a chromatographic, e.g., HPLC, purification procedure. However, this technique requires specialized equipment and can moreover be tedious and time-consuming. Considering the half-life of most clinically useful radioisotopes, it is desirable to complete the radiosynthesis and purification prior to administration to a patient as rapidly as possible. For example, the half-life of ¹⁸F is 110 minutes and ¹⁸F-labeled targeting vectors are therefore synthesized and purified within one hour of clinical use.

In view of the above, it is readily apparent that there is a need in the art for purification techniques which offer rapid and efficient separation of unwanted species from the final pharmaceutical vector-X.

Since the introduction of the Merrifield method for peptide synthesis, insoluble polymer supports have been incorporated into numerous synthetic methodologies to facilitate product purification. In the solid phase peptide synthesis method, the nucleophile of a substitution reaction is covalently linked to a solid phase resin as shown in Scheme 2a. Following the substitution reaction, the excess vector-L and the displaced leaving group L are easily separated from the resin-bound product Resin-X-vector by filtration.

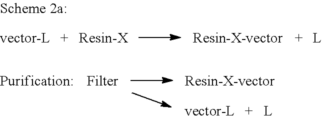

WO 2003/0012730 discloses an alternative radiohalogenation method in which the vector of the substitution reaction is covalently linked to a solid phase resin through the leaving group as shown in Scheme 3a.

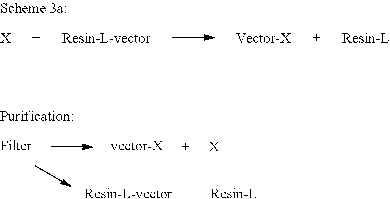

With this strategy, a radiolabelling agent X is reacted with the solid phase-supported vector to form vector-X, which is conveniently separated from non-reacted Resin-L-vector and resin-bound leaving group Resin-L by washing and filtering off the resin.

Solid phase processes for the production of ¹⁸F-radiolabeled tracers suitable for use as positron emission tomography radiotracers are for example disclosed in WO 2003/002157.

Although solid phase-supported nucleophilic substitution technologies can simplify purification steps substantially, they suffer from the inherent drawback that heterogeneous reaction conditions are usually less efficient, leading to poor radiochemical yields and slower reaction times compared to reactions carried out in solution, i.e., without a solid support.

Alternative radiolabelling strategies that utilize homogeneous substitution reaction conditions are for example disclosed in WO 2005/107819 and in a scientific publication of Donavan et al (J. Am. Chem. Soc., 2006, 128, 3536-3537).

WO 2005/107819 relates to the purification of a radiolabeled tracer vector-X-R* resulting from a substitution reaction of R* for Y on the substrate vector-X-Y, using a solid support-bound scavenger group (scavenger resin). The scavenger resin Z-resin undergoes a similar substitution reaction on the non-reacted substrate vector-X-Y to displace Y and generate vector-X-Z-resin, which can be filtered off from the product vector-X-R* (which remains in solution). Hence, the purification procedure separates product from unreacted precursor. The scavenger resins are only designed to displace the moiety Y of the reactive group. In other words, this approach is limited to remove non-reacted precursors but does not allow a simultaneous removal of Y leaving group from the product. Furthermore, the reactive moiety Z of the scavenger resin described in WO 2005/107819 is limited only to groups that are good substitution agents for Y.

Donavan et al. (loc. cit.) describe a “homogeneous” soluble supported procedure for electrophilic radioiodine substitution utilizing a fluorine-rich soluble support wherein a leaving group is linked to a perfluorinated moiety. The radioiodinated product was isolated from both unreacted substrate and the leaving group based on the strong affinity of the perfluorinated moiety for other perfluorinated species.

Although this homogeneous substitution procedure with fluorous-based purification has been demonstrated effective for radioiodination, it is not likely to be as useful for, e.g., ¹⁸F radiolabelling or nucleophilic reactions in general, because the Sn substrates are specific for electrophilic substitutions. Electrophilic ¹⁸F substitution is not often performed because the radiofluorine gas [¹⁸F] F₂ is not readily available, and because it has low specific activity (resulting from added [¹⁹F] F₂ carrier gas). Furthermore, fluorous-based purification using more preferred (higher specific activity) nucleophilic substitution reactions with [¹⁸F] fluoride are expected to be problematic in view of the exchange of ¹⁸F for cold (¹⁹F) fluorine from the perfluorinated moiety. Such fluorine exchange reactions are well-known, and can lead to lower radiochemical yields and poor specific activity of the radiopharmaceutical.

From the above, it is evident that other soluble-supported purification strategies for inter alia radiohalogen pharmaceuticals are needed which are easy to use and provide a broader applicability compared to the prior art described above. It would therefore be useful to develop alternative strategies for purifying, e.g., radiohalogen-containing pharmaceuticals which do not require HPLC purification and moreover reliably ensure efficient separation of vector-X from unreacted precursor compounds vector-L as well as from the leaving group by-product L.

### SUMMARY OF THE INVENTION

The present invention generally relates to novel processes and kits for the preparation and purification of pharmaceuticals. In particular, this invention relates to methods and kits for carrying out an efficient liquid phase nucleophilic substitution reaction for preparing pharmaceuticals, including radiopharmaceuticals, and to a subsequent purification of the product using the leaving group whereby the lipophilicity of this leaving group has been modified to allow for easier, simpler purifications. The purification process of the present invention separates the substitution product from non-reacted precursor molecules and from displaced leaving groups of a nucleophilic substitution reaction.

The processes and the products they produce are advantageous in several respects. The processes allow a simple and effective separation of non-reacted precursors and by-products from the desired main product using standard laboratory manipulations and without the need for sophisticated purification equipment. In addition, the separation procedures as described herein according to the method of the present invention are often much more convenient, flexible and most importantly less time consuming, which is a great advantage for example in the handling of clinically employed short-lived radiopharmaceuticals such as ¹⁸F-labeled pharmaceuticals.

Accordingly, the present invention relates in a first aspect to a process for preparing a pharmaceutical vector-X, wherein the moiety L^{M} of a precursor species vector-L^{M} is replaced by a reactant X through a liquid phase nucleophilic substitution to form said pharmaceutical vector-X and a species L^{M}, wherein vector is a targeting vector; L^{M} is a leaving group with modified lipophilicity covalently attached to vector prior to said nucleophilic substitution reaction; the characteristics of L^{M} that allow for simpler purification methods compared to species that do not contain said modified leaving group L^{M}. Optionally, vector-X is further reacted to yield the final product vector-X′.

In a second aspect, the invention relates to a process for preparing and purifying a pharmaceutical vector-X, wherein the moiety L^{M} of a precursor species vector-L^{M} is replaced by a reactant X through a liquid phase nucleophilic substitution to form said pharmaceutical vector-X and a leaving group species L^{M}, optionally, wherein vector-X is further reacted to yield the final product vector-X′; and wherein any species that still contain said modified leaving group L^{M} are selectively separated from species not containing said modified leaving group L^{M}, preferably vector-X, by a purification procedure, e.g., as set out herein in further detail below.

In a third aspect, the present invention relates to a process for purifying a pharmaceutical vector-X from a liquid phase reaction mixture comprising vector-X, vector-L^{M}, and optionally L^{M} by selectively separating any species which contain said modified leaving group L^{M} from said pharmaceutical vector-X using a purification procedure. Suitable purification procedures according to the present invention will be described in further detail hereinbelow.

In preferred embodiments, the liquid phase nucleophilic substitution reaction is a homogeneous nucleophilic substitution reaction, i.e. the reaction is carried out in a single liquid phase.

Furthermore, another aspect of the present invention relates to kits for carrying out a nucleophilic substitution and/or purification according to the present invention. In one embodiment, a kit according to the invention comprises at least a modified leaving group L^{M} to be attached to vector. Optionally, kits according to the present invention comprise a product manual, one or more compounds or resins to carry out a purification step and/or suitable reaction or purification media and the like.

### BRIEF DESCRIPTION OF THE FIGURES

FIG. 1: TLC of four different sulfonyl chlorides in normal phase and reverse phase. Ts=Tosyl Chloride; Cs=Cesyl Chloride (6); Ds=Dipsyl Chloride (7); Chs=Cholesyl Chloride (8).

FIG. 2: HPLC purification of [¹⁸F]FLT and precursor Nosylate-FLT wherein Nosylate leaving group shows big organic impurities peak at before & after true [¹⁸F]FLT peak.

### DETAILED DESCRIPTION

First Aspect:

The present invention relates to nucleophilic substitution reactions carried out under liquid phase, preferably homogeneous, reaction conditions, i.e., the substitution takes place in liquid reaction media. The novel liquid phase nucleophilic substitution and subsequent purification processes of the present invention are shown in a generalized manner in Scheme 4a below.

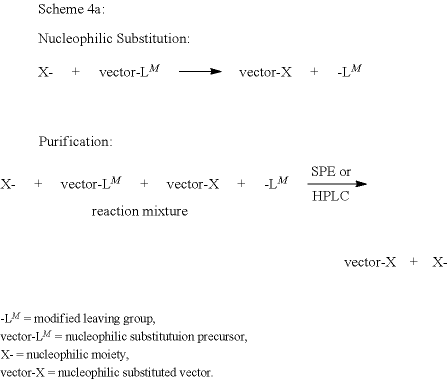

The invention relates to a method of preparation of compound of Formula II by direct nucleophilic radiofluorination of compound of Formula I

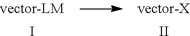

wherein
the difference between the logD of compound of Formula I and the logD of compound of Formula II is greater than 1.5,
the vector is a targeting vector,
L^{M} is a modified leaving group, suitable for direct nucleophilic fluorination, and
X is a nucleophilic moiety.
Preferably the nucleophilic moiety X comprises radiohalogen isotope wherein the radiohalogen isotope is preferably ¹⁸F.
Preferably, the difference between the logD of compound of Formula I and the logD of compound of Formula II is greater than 2, more preferably greater than 4.
Preferably, L^{M} is a sulfonate derivative.
More preferably, L^{M} is

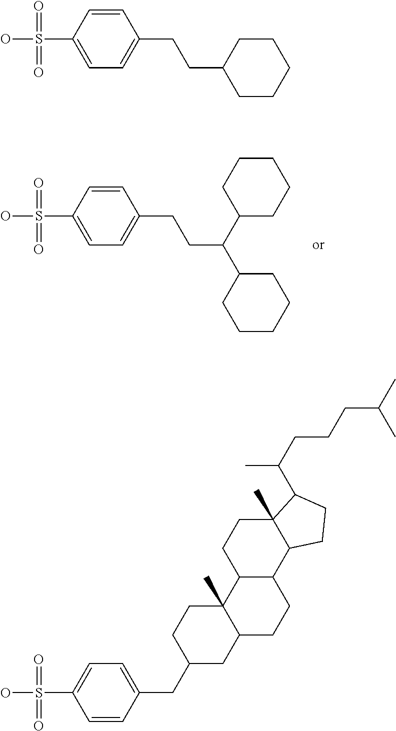

Preferably the compound of Formula I is selected from the group comprising

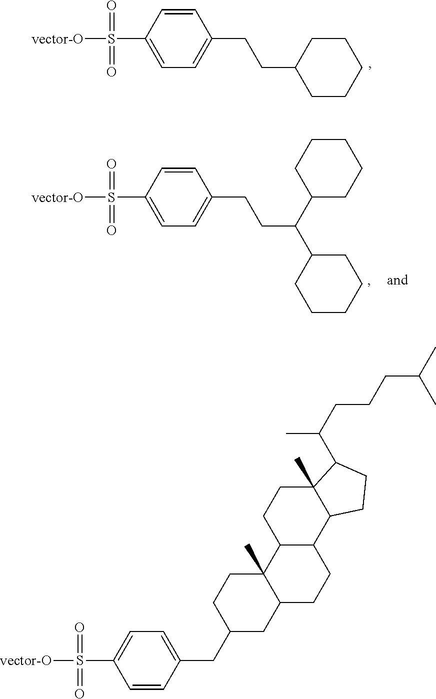

Although the preferred embodiments of the present invention refer to nucleophilic substitutions with radioactive halogen-isotopes such as ¹⁸F, any such references to radiohalogens are used by way of example only and are not intended to be limiting in any way. For example, the process can also be carried out to produce other radiopharmaceuticals, halogen-containing non-radioactive pharmaceuticals or even any nucleophilic residue-containing pharmaceuticals.

All processes of the present invention are characterized by the involvement of a special modified leaving group (L^{M}). In accordance with the present invention, said modified leaving group L^{M} is covalently linked to a vector for forming a precursor compound vector-L^{M} that is subjected to a nucleophilic substitution reaction to attach a nucleophilic moiety X, which, e.g., may be derived prior or during the substitution reaction from a precursor X* (X* being a suitable precursor providing the nucleophile X to the reaction: A non-limiting example for, e.g., X* is a salt of X) or from a precursor X**, wherein X is a nucleophilic moiety that is transferred from X** to vector during the nucleophilic reaction. In the nucleophilic substitution reactions (and the kits) of the present invention, vector is a targeting vector and L^{M} is a leaving group during said nucleophilic substitution reaction.

The modified leaving group L^{M} has characteristics due to the increased lipophilicity that allow any species that contain L^{M} to be easily separated from other species that do not contain L^{M}. These leaving groups with increased liphophilicity are clearly more lipophilic than the leaving groups used by those skilled in the art, i.e. mesylate, triflate or tosylate. Various separation procedures that are effected by employing the modified leaving group L^{M} are described in further detail hereinbelow. The modified leaving group L^{M} allows the efficient and convenient separation of non-reacted precursors vector-L^{M} and the by-product L^{M} from the desired product vector-X. It will be appreciated that the separation of L^{M}-containing species from those that do not contain L^{M} depends on the extend of lipophic properties of L^{M} and can generally be performed by methods known to those skilled in the art. While not limited to these embodiments, the present invention is illustrated by describing a variety of separation types in more detail.

Second Aspect:

The invention is related to a method for separating a compound of Formula II from compound of Formula I and side products resulting from or participating to the nucleophilic substitution reaction as in the first aspect

wherein

vector-L^{M} I
that is a precursor for a direct nucleophilic radiofluorination compound of Formula II and

vector-X II
wherein
the difference between the logD of compound of Formula I and the logD of compound of Formula II is greater than 1.5, vector is a targeting vector, X is a nucleophilic moiety and L^{M} is a modified leaving group suitable for direct nucleophilic fluorination.
   Side products are compound containing L^{M} (vector-L^{M}) or L^{M} as such.

The method useful for separating 2 species is selected from the group of solid-phase-extraction, filtration, precipitation, distillation and liquid-liquid-extraction.

Preferably, the difference between the logD of compound of Formula I and the logD of compound of Formula II is greater than 2, more preferably greater than 4.
Preferably, L^{M} is a sulfonate derivative, see above for more detail.
Preferably, the nucleophilic moiety X comprises radiohalogen isotope wherein the radiohalogen isotope is preferably ¹⁸F.
Preferably, the method for separating comprises the step of
contacting the mixture of compounds of Formula I and X with a liquid or solid phase having a high affinity for L^{M}, and
removing compound of Formula II by liquid extraction phase.
   Additionally, the method is optionally preceded by the method of first aspect (method of preparation of compound of Formula II by direct nucleophilic radiofluorination of compound of Formula I.

Separation is based on liquid-liquid or solid-liquid extraction using a solution phase (liquid phase) or a resin (solid phase) that have affinity to L^{M}. In such separations, the removal of an L^{M}-containing species into a liquid extraction phase or to a solid resin generally relies on the affinity of a L^{M} to the polar, ionic, or non-polar properties of the liquid extraction phase or solid resin. In general, any species that do not contain said moiety L^{M} (such as the desired reaction product vector-X) essentially remains in the reaction mixture and is not transferred to the liquid extraction phase or solid resin, thereby achieving a separation of L^{M}-containing species from those that do not contain L^{M}. Alternatively, in embodiments of liquid-liquid extraction, L^{M}-containing species may have an affinity to the reaction mixture and essentially remain in said mixture, i.e., they are not transferred to the liquid extraction phase.

In other embodiments of the liquid-liquid extraction described above, the separation of L^{M}-containing species and species that do not contain a moiety L^{M} is based on the affinity of L^{M}-containing species to the liquid reaction phase whereas species that do not contain a purification moiety L^{M} are extracted into a liquid extraction phase, i.e. in a particular embodiment of liquid-liquid extraction separation methods, L^{M} has an affinity for the reaction solution liquid phase rather than for the extracting liquid phase, and therefore the reaction product vector-X can be extracted from the reaction solution to effect the purification.

In other words, in certain embodiments of the present invention related to the separation of L^{M}-containing species from species that do not contain a moiety L^{M}, the separation relies on the affinity of said L^{M} to the reaction phase.

In embodiments of solid-liquid extraction, the extraction of L^{M}-containing species relies on the affinity of said species to a solid resin (or to a group that is attached to a resin).

Furthermore, L^{M}-containing species can also be separated from the product vector-X, i.e. they can be removed from the reaction mixture, by precipitation and subsequent filtration or centrifugation because L^{M} makes them prone to precipitate under certain conditions (Separation Type B). For example, L^{M} can contain a cholesteryl moiety, which are prone to precipitate when added to water. Such compounds can then be easily removed by filtration or centrifugation.

Preferred features for X, L^{M}, vector-L^{M} in the first aspect are enclosed herein.

Third Aspect:

The invention is related to a compound of Formula I:

vector-L^{M} I
that is a precursor for a direct nucleophilic radiofluorination compound of Formula II:

vector-X II
wherein
the difference between the logD of compound of Formula I and the logD of compound of Formula II is greater than 1.5, vector is a targeting vector, X is a nucleophilic moiety and
L^{M} is a modified leaving group suitable for direct nucleophilic fluorination.
   Preferably the difference of logD of compound of Formula I and the logD of compound of Formula II is greater than 2, more preferably greater than 4.
   Preferred features can be combined together and are within the scope of the invention, see above.
   The following compounds are selected compounds of the invention

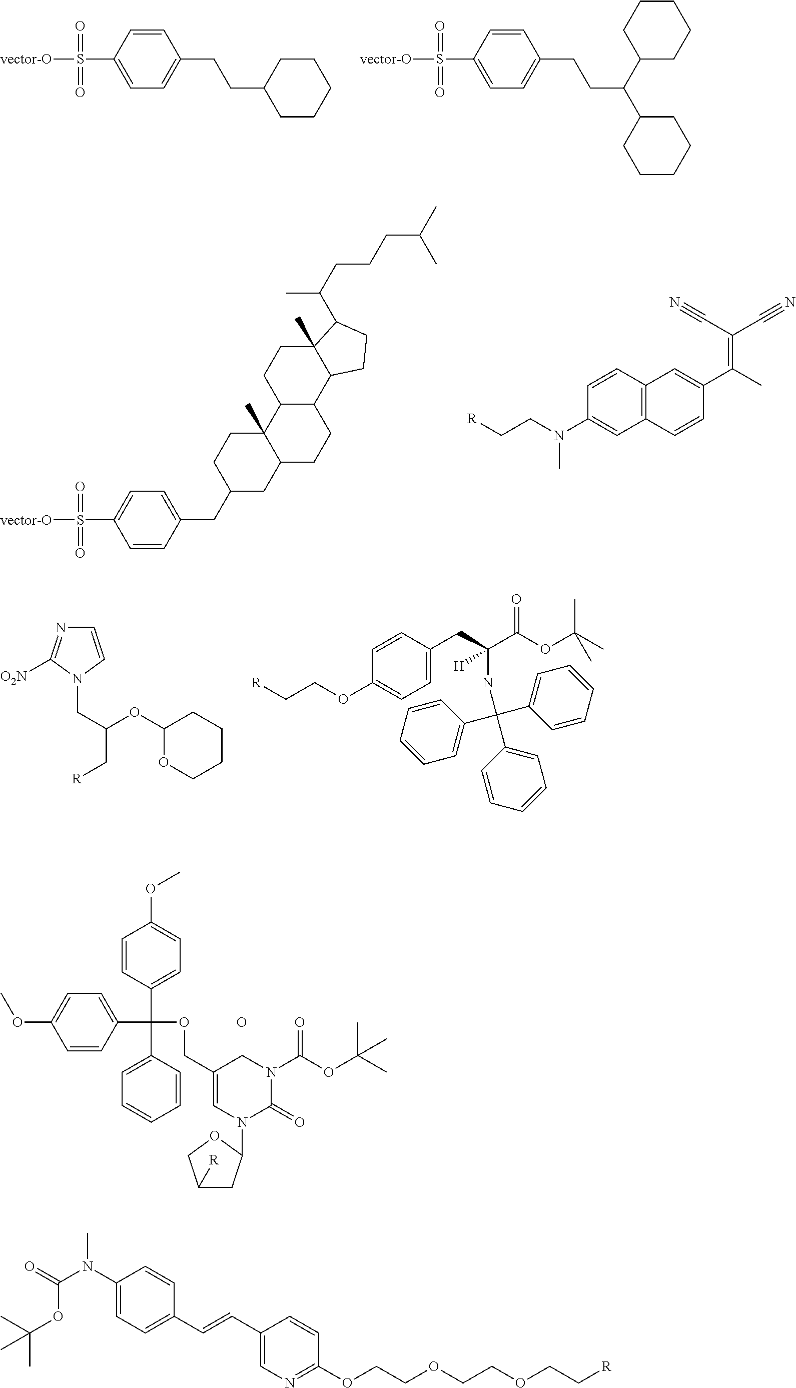

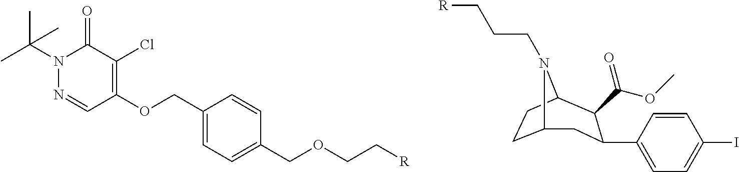

wherein R is

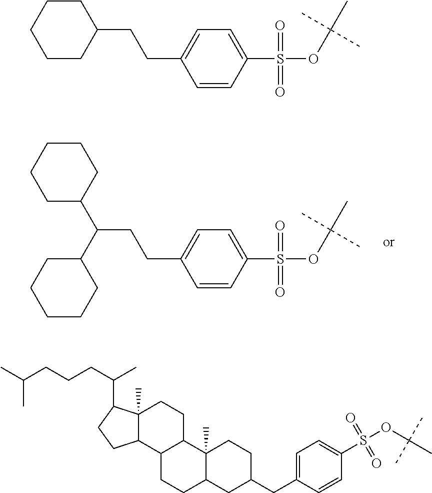

Fourth Aspect:

The invention is related to a compound of Formula III

R1-L^{M1} (III)

wherein
R1 is halide and covalently bound to S* and

### L^{M1} is

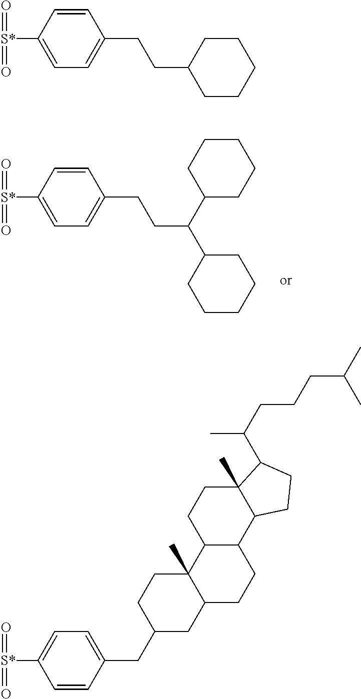

Preferred compound are
3-[(10S,13R)-17-(1,5-Dimethyl-hexyl)-10,13-dimethyl-hexadecahydro-cyclopenta[a]phenanthren-3-ylmethyl]-benzenesulfonyl chloride (3)-Cholesyl Chloride

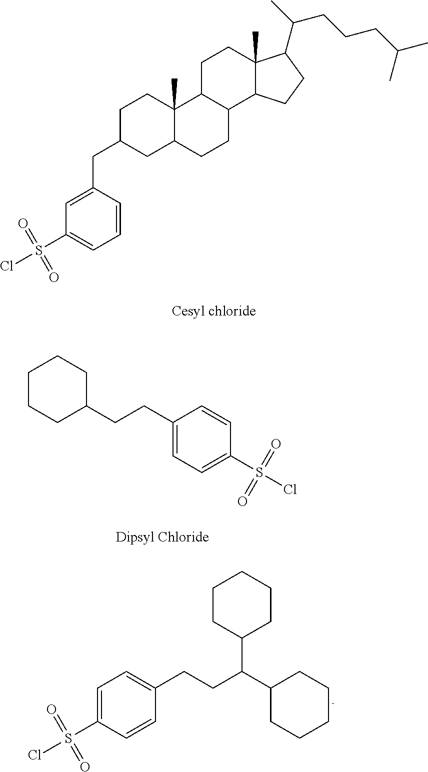

Fifth Aspect:

The invention is related to a method for obtaining a compound of formula I by reacting a compound of formula III with a vector.

Preferred features for R1, L^{M}, vector-L^{M} (compound of formula I) in the first aspect are enclosed herein.

### DEFINITIONS

In the context of the present invention, the following definitions shall apply:

The terms “a” or “an” as used herein means “one”, “at least one” or “one or more”.

The nucleophilic substitution reaction according to the present invention is carried out in a “liquid phase”. A liquid phase nucleophilic substitution reaction as defined herein refers either to a two phase liquid-liquid reaction, e.g., two non-miscible solvents, optionally in the presence of a phase transfer catalyst, or it refers to a “homogeneous reaction”. The term “homogeneous” as used herein to describe a substitution reaction means that the reaction conditions are uniform (i.e. in contrast to a heterogeneous reaction, as, e.g., described for the prior art purifications involving solid supports). In other words, the homogeneous nucleophilic substitution reaction takes place in a single liquid phase and the reactants are dissolved within said phase during the reaction. The person skilled in the art will understand that some compounds may precipitate from the liquid reaction mixture after completion of the substitution reaction, but the latter is not to be confused with a heterogeneous nucleophilic reaction.

The term “vector” or “targeting vector” as used herein describes a compound that preferably possesses inherent properties that give it a biodistribution favorable for imaging a pathology, disease or condition. Prior to the nucleophilic substitution reaction, the vector is covalently linked to a modified leaving group L^{M} is subjected to a liquid phase nucleophilic substitution reaction.

Vector can be any suitable targeting vector chosen for the intended purpose and has generally a molecular weight of less than about 50000, about 30000, about 15000, about 10000, preferably less than about 5000 Da, more preferably less than about 2500 Da and most preferably less than about 1500 Da.

It is readily apparent that already for practical reasons, small targeting vectors are preferred, not the least because the chemistry is better defined and there are generally less functional groups that may interact/interfere with the nucleophile X in the liquid phase nucleophilic substitution reaction of the present invention. The vector is typically selected from the group consisting of a synthetic small molecule, a pharmaceutically active compound (i.e., a drug molecule), a metabolite, a signaling molecule, an hormone, a peptide, a protein, a receptor antagonist, a receptor agonist, a receptor inverse agonist, a vitamin, an essential nutrient, an amino acid, a fatty acid, a lipid, a nucleic acid, a mono-, di-, tri- or polysaccharide, a steroid, and the like. It will be understood that some of the aforementioned options will overlap in their meaning, i.e., a peptide may for example also be a pharmaceutically active compound, or a hormone may be a signaling molecule or a peptide hormone. Furthermore, it will be understood that also derivatives of the aforementioned substance classes are encompassed.

The vector (or, optionally, any metabolite of the vector or vector-X, respectively), is preferably a moiety that specifically binds to a target site in a mammalian body. Specific binding in this context means that the vector, or vector-X for that matter, accumulates to a larger extent at this target site compared to the surrounding tissues or cells. For example, the vector may specifically bind to a receptor or integrin or enzyme that is preferentially expressed at a pathologic site within the mammalian body, or the vector may be specifically transported by a transporter that is preferentially expressed at a pathologic site within the mammalian body. In some embodiments, the receptor, integrin, enzyme, or transporter is exclusively expressed at a pathologic site within the mammalian body, i.e., to sites that are different or absent in healthy subjects, or vice versa. In this context, it will be understood that the vector preferably binds specifically to a receptor/or integrin/or enzyme/or transporter that is exclusively expressed or present at a pathologic site within the mammalian body and not expressed or present at a non-pathologic site, although the latter is—while no doubt highly desirable-rarely achieved in practice.

Examples for specific binding include, but are not limited to, specific binding to a site of infection, inflammation, cancer, platelet aggregation, angiogenesis, necrosis, ischemia, tissue hypoxia, angiogenic vessels, Alzheimer's disease plaques, atherosclerotic plaques, pancreatic islet cells, thrombi, serotonin transporters, neuroepinephrin transporters, LAT 1 transporters, apoptotic cells, macrophages, neutrophils, EDB fibronectin, receptor tyrosine kinases, cardiac sympathetic neurons, and the like.

In preferred embodiments, the vector may be selected from the group consisting of a synthetic small molecule, a pharmaceutically active compound (drug), a peptide, a metabolite, a signaling molecule, a hormone, a protein, a receptor antagonist, a receptor agonist, a receptor inverse agonist, a vitamin, an essential nutrient, an amino acid, a fatty acid, a lipid, a nucleic acid, a mono-, di-, tri-, or polysaccharide, a steroid, a hormone and the like. More specifically, the vector may be selected from the group consisting of glucose, galactose, fructose, mannitol, sucrose, or stachyose and derivatives thereof (e.g. N-Ac groups are attached or functional groups other than -L^{M} are protected), glutamine, glutamate, tyrosine, leucine, methionine, tryptophan, acetate, choline, thymidine, folate, methotrexate, Arg-Gly-Asp (RGD) peptides, chemotactic peptides, alpha melanotropin peptide, somatostatin, bombesin, human pro-insulin connecting peptides and analogues thereof, GPIIb/IIIa-binding compounds, PF4-binding compounds, ανβ3, ανβ6, or α4β1 integrin-binding compounds, somatostatin receptor binding compounds, GLP-1 receptor binding compounds, sigma 2 receptor binding compounds, sigma 1 receptor binding compounds, peripheral benzodiazepine receptor binding compounds, PSMA binding compounds, estrogen receptor binding compounds, androgen receptor binding compounds, serotonin transporter binding compounds, neuroepinephrine transporter binding compounds, dopamine transporter binding compounds, LAT1 transporter binding compounds and hormones such as peptide hormones, and the like.

In embodiments of the present invention, it will generally be preferred that the vector-X shows essentially the same biologically relevant features, e.g., being a targeting moiety that specifically binds to a target site in a mammalian body, as the vector. In other words, X essentially does not alter the targeting properties of the vector.

Furthermore, in another preferred embodiment, vector-X may accumulate in a major organ in a manner that allows estimation of regional tissue perfusion in that organ. For example, the vector may accumulate in the heart of a potential heart attack patient according to regional perfusion levels, and allow for delineation of areas where the heart has obstructed coronary arteries. Similarly, the vectors reflecting perfusion in the brain could help identify areas of stroke.

The term “protein”, as used herein, means any protein, including, but not limited to peptides, enzymes, glycoproteins, hormones, receptors, antigens, antibodies, growth factors, etc., without limitation, having at least about 20 or more amino acids (both D and/or L forms thereof). Included in the meaning of protein are those having more than about 20 amino acids, more than about 50 amino acid residues, and sometimes even more than about 100 or 200 amino acid residues.

The term “peptide” as used herein refers to any entity comprising at least one peptide bond, and can comprise either D and/or L amino acids. The meaning of the term peptide may sometimes overlap with the term protein as defined herein above. Thus, peptides according to the present invention have at least 2 to about 100 amino acids, preferably 2 to about 50 amino acids. However, most preferably, the peptides have 2 to about 20 amino acids, and in some embodiments between 2 and about 15 amino acids.

The term “small molecule” is intended to include all molecules that are less than about 1000 atomic units. In certain embodiments of the present invention, the small molecule is a peptide which can be from a natural source, or be produced synthetically. In other embodiments, the small molecule is an organic, non-peptidic/proteinaceous molecule, and is preferably produced synthetically. In particular embodiments, the small molecule is a pharmaceutically active compound (i.e., a drug), or a prodrug thereof, a metabolite of a drug, or a product of a reaction associated with a natural biological process, e.g., enzymatic function or organ function in response to a stimulus. small molecule has generally a molecular weight of between about 75 to about 1000.

Non-limiting examples for a peptide hormone are: angiotensin, leptin, prolaktin, oxytocin, vasopressin, bradykinin, desmopressin, gonadoliberin, insulin, glucagon, gastrin, somatostatin, calcitonin, parathormon, ANF, ghrelin, obestatin, HCG, thyreotropin, thyreoliberin, follitropin, luteotropin, adrenocortikotropin, MSH, EPO, somatotropin, IGF, LH/FSH, TSH, ACTH and GH.

Because the vector is generally comprised within a vector-L^{M} species, it will be understood that the vector refers to any form of the vector being suitable to take part in a selective nucleophilic substitution reaction to exchange the modified leaving group L^{M} (attached to the vector) against a nucleophilic agent X or a moiety/molecule/precursor comprising X. In other words i.e., the vector may optionally possess other reactive groups in addition to L^{M}. In some embodiments, at least one of said other reactive groups has to be protected before the nucleophilic substitution is carried out. Additionally, the vector may be a precursor of the desired pharmaceutical, i.e., the vector has to be further modified after the nucleophilic substitution to obtain the desired product.

The leaving group L^{M} is preferably selected from the group consisting of —OSO₂—R, where R has been modified to enable simpler purification methods.

In certain embodiments, a leaving group L^{M} contains more than one R.

The term “L^{M}” or “modified leaving group” as used herein refers to a moiety that is associated with the nucleophilic substitution reaction and is covalently bound to the vector and is substituted by said nucleophilic reactant X. L^{M} as defined herein has characteristics that allow species that contain said purification moiety L^{M} to be separated from other species that do not contain said purification moiety L^{M}.

The terms “X” or “reactant X” as used herein refer to any nucleophilic agent suitable to perform a nucleophilic substitution of the L^{M} moiety of a vector-L^{M} precursor resulting in a vector-X species. For example, X is a nucleophilic agent in its entirety or is a moiety/molecule comprising a nucleophilic group that reacts with the vector-L^{M} (e.g. an amine group). Alternatively, X may be derived from a precursor X* (e.g., a salt), or from a precursor X**, wherein X is a nucleophilic moiety that is transferred from X** to the vector during the nucleophilic reaction and wherein X thereby substitutes the moiety L^{M} of the vector-L^{M}. The reactive region of a reactant X is preferably negatively charged, but may also be a polar electron rich part of the molecule. As apparent from the foregoing, the terms “X” or “reactant X” as used herein are meant to describe all possible forms of X that may, e.g., be present in a reaction mixture to perform a nucleophilic reaction in accordance with the present invention prior to, during and after said reaction. As an example for illustrating the different possible forms of X useful in the context of the present invention, X can be an anionic form prior to said nucleophilic reaction, or can be comprised in an intermediate state during the nucleophilic reaction, and will in any case be a covalently attached group to the vector after the nucleophilic reaction forming the product vector-X. It will be understood that the same principle extends also to the other synonyms of the species used herein, e.g., the vector, L^{M}, etc.

In particular embodiments of the present invention, X is a halogen or a halide, for example fluorine or fluoride. In other preferred embodiments, the nucleophilic agent X is or comprises a radioisotope selected from but not limited to the group of ^{99m}Tc, ¹¹¹In, ¹⁸F, ²⁰¹Tl, ¹²³I, ¹²⁴I, ¹²⁵I, ¹³¹I, ³⁴Cl ¹¹C, ³²F, ⁷²As, ⁷⁶Br, ⁸⁹Sr, ¹⁵³Sm, ¹⁸⁶Re, ¹⁸⁸Re, ²¹²Bi, ²¹³Bi, ⁸⁹Zr, ⁸⁶Y, ⁹⁰Y, ⁶⁷Cu, ⁶⁴Cu, ¹⁹²Ir, ¹⁶⁵Dy, ¹⁷⁷Lu, ¹¹⁷Sn, ²¹³Bi, ²¹²At, ²¹¹At, ²²⁵Ac, ²²³Ra, ¹⁶⁹Yb, ⁶⁸Ga and ⁶⁷Ga.

It will be appreciated that some of the radioisotopes listed above are not suitable to perform a nucleophilic substitution reaction on their own. Those of skill in the art will, however, know which of the listed radioisotopes may be suitable to represent the nucleophile in a nucleophilic reaction (such as ¹⁸F), and which radioisotopes have to be bound to another nucleophilic moiety suitable to substitute L^{M} by virtue of a nucleophilic substitution reaction.

In embodiments wherein X is a radioisotope, it is preferred if the radioisotope is a radiohalogen such as ¹⁸F, ¹²³I, ¹²⁴I, ¹²⁵I, ¹³¹I, ³⁴Cl and ²¹¹At. The most preferred radioisotope in the context of the present invention is the ¹⁸F radioisotope of fluorine. It will be understood that the radiohalogens listed above may be present in a reaction mixture as nucleophilic agents (i.e. as an anionic species or as a polar moiety, etc.) or they may be comprised within a nucleophilic agent, wherein the radioisotope is not actively involved in the nucleophilic reaction but is a part of the substituting moiety X.

Typically, and if not explicitly stated otherwise, the term “precursor” as used herein refers to at least one, more than one or all reactants of the nucleophilic substitution reaction, i.e., X, X*, X** and vector-L^{M}.

The term “vector-X” as used herein relates to the product of a liquid phase nucleophilic reaction of a precursor/reactant vector-L^{M} with a nucleophilic agent X. The term “vector-X” encompasses neutral, non-polar, polar, negatively or positively charged species. The product “vector-X” comprises protected reactive groups and/or may be subjected to further modifications that do not interact with the vector-X bond such as, e.g., deprotection or modifying a different reactive group to prepare a final product vector-X′.

In one preferred embodiment, vector-X is a halogenated product. In yet another preferred embodiment, vector-X is a radiolabeled product, preferably a radiohalogen labeled product and most preferred an ¹⁸F labeled product.

The term “reaction medium” as used herein typically comprises all compounds such as buffers, salts, solvents, and soluble supports to perform a nucleophilic reaction according to the present invention. It is to be understood that, optionally, the precursors vector-L^{M} and X may additionally be present in a reaction medium prior to the nucleophilic substitution.

The term “reaction mixture” as used herein refers typically to a liquid composition which is subjected to a liquid phase nucleophilic substitution reaction according to the present invention and comprises, or is suspected of comprising, a main product and optionally by-products and non-reacted reactants. A reaction mixture may comprise additives, which are added after said substitution reaction and prior to a subsequent purification step, to create conditions more suitable to perform said purification step, e.g., slightly changing the pH by adding an acid or a base to obtain an optimized pH value for, e.g., a chelating solid-liquid extraction.

In the context of the present application, the terms “to purify”, “purification”, “to separate” and “separation” are used interchangeably and are intended to mean any partitioning of a mixture of two or more species based on the presence or absence of a purification moiety L^{M}, wherein at least one species that does not contain a moiety L^{M} remains in or is extracted into a liquid fraction and the L^{M}-containing species end up in a separate liquid or a solid fraction. Separation therefore includes, but is not limited to, a specific and selective enrichment or depletion, concentration and/or isolation of L^{M}-containing species, or vice versa, of a species that does not contain a moiety L^{M}. However, it will be appreciated that purifying is typically understood to mean a depletion of O-containing species within a liquid phase which also containing a species that does not contain a moiety L^{M} (, regardless of whether said non-L^{M} species that does not contain a moiety L^{M} is further modified or separated from other compounds). It is readily apparent that there may be impurities of L^{M}-containing species left in a liquid phase after the purification.

Therefore, it is to be understood that “to purify” as used herein relates to a depletion of L^{M}-containing species in a liquid phase containing at least one species that does not contain a moiety L^{M} of at least 40%, of at least 50%, of at least 60%, of at least 70%, of at least 80%, or of at least 90% after a purification step, although the term preferably means an even more complete depletion of the L^{M}-containing species. Thus, whenever the application refers to the terms “to purify”, “purification”, “to separate” or “separation”, they are intended to relate to a depletion of L^{M}-containing species from a liquid phase containing a species that does not contain a moiety L^{M} after a purification step of 50%, 60%, 70%, 80%, 90%, preferably 95%, more preferably 99% and most preferably 100%. In cases where the purification level is not at least 95%, preferably 97%, more preferably 99% and most preferably 100% depletion of L^{M}-containing species, serial (repeat) purification procedures may be carried out on with the reaction mixture to increase the overall purification to the desired level.

The terms “soluble supported” or “soluble support” as used herein refer to a method of synthesis on soluble polymers such as polyethylene glycol. In contrast to “classical” or “solution” synthesis which refer to homogeneous reaction schemes that do not employ polymer supports, the term “soluble supported” reactions as used herein are reserved for methodologies incorporating a soluble macromolecular carrier to facilitate, e.g., product isolation.

“Soluble supports” according to the present invention are soluble macromolecular carriers. Typically, soluble supports suitable for methods of the present invention demonstrate good chemical stability and provide appropriate functional groups for easy attachment of organic moieties and exhibit solubilizing power in order to dissolve molecular entities with low solubilities and permit a general synthetic methodology independent of the physicochemical properties of target compounds. It is to be understood that soluble supports may typically exhibit not one discrete molecular weight but instead may consist of macromolecules with variable sizes/molecular weights. Suitable solid supports can be selected from but are not limited to the group consisting of polystyrene, polyvinyl alcohol, polyethylene imine, polyacrylic acid, polymethylene oxide, polyethylene glycol, polypropylene oxide, cellulose, polyacrylamide and the like.

The term “resin” as used herein refers to a solid phase, i.e. it is insoluble in the liquid used for carrying out the nucleophilic substitution reaction or during subsequent purification. Typically, a resin is a polymer, which may optionally comprise reactive groups that are attached to the surface of the resin or that are attached to the surface of the resin by a linker.

As will be appreciated from the foregoing, the present invention is inter alia directed to methods for preparing pharmaceuticals comprising a liquid-phase nucleophilic substitution reaction, and possible downstream methods to purify the desired product from unreacted reactants.

The term “halide” as employed herein by itself or as part of another group is known or obvious to someone skilled in the art, and means fluoro, chloro, bromo, and iodo.

### Methods

One embodiment is a process for preparing a pharmaceutical vector-X, wherein the moiety L^{M} of a precursor species vector-L^{M} is replaced by a reactant X through a liquid phase nucleophilic substitution to form said pharmaceutical vector-X and a species L^{M}, wherein
vector is a targeting vector
L^{M} is a modified leaving group covalently attached to the vector prior to said nucleophilic substitution reaction; and
   optionally, wherein vector-X is further reacted to yield the final product vector-X′.

Another aspect of the present invention relates to a process for preparing and purifying a pharmaceutical vector-X, wherein the moiety L^{M} of a precursor species vector-L^{M} is replaced by a reactant X through a liquid phase nucleophilic substitution to form said pharmaceutical vector-X and a species L^{M}, wherein
vector is a targeting vector;
L^{M} is a modified leaving group covalently attached to the vector prior to said nucleophilic substitution reaction; and
   optionally, wherein vector-X is further reacted to yield the final product vector-X′; and
   wherein any species which still contain said purification moiety L^{M} (L^{M}-containing species) are selectively separated from species not containing said purification moiety L^{M}, preferably vector-X, by using a purification procedure.

Yet another aspect of the present invention relates to a process for purifying a pharmaceutical vector-X from a liquid phase reaction mixture comprising vector-X, vector-L^{M}, and optionally L^{M}, wherein;
vector is a targeting vector;
L^{M} is a modified leaving group covalently attached to the vector prior to said nucleophilic substitution reaction; and
   by selectively separating any species which contain said moiety L^{M} from vector-X using a purification procedure.

In particular embodiments of the present invention, the nucleophilic substitution reaction of vector-L^{M} to S-X is carried out as a soluble supported reaction.

In preferred embodiments of the present invention, the nucleophilic reaction of vector-L^{M} to vector-X is carried out as a homogeneous nucleophilic substitution reaction.

The separation of a species that lacks a moiety L^{M}, such as the desired product vector-X, from one or several L^{M}-containing species may be carried out using methods generally known to a person skilled in the art. Suitable examples will be described in more detail in the following section.

### Purification Methods

### Liquid-Liquid Purification

In preferred embodiments, species that do not contain the modified leaving group L^{M} can be separated from L^{M}-containing species by liquid-liquid phase extraction. Thus, L^{M}-containing species can, e.g., be removed from the reaction mixture. Alternatively, non-L^{M}-containing species (e.g. vector-X) can be removed from the reaction mixture and the L^{M}-containing species essentially stay within the reaction mixture.

Those of skill in the art are generally aware of the principles of liquid-liquid extraction. Preferably, a liquid-liquid extraction according to the present invention relies on the lipophilicity of the moiety L^{M} to the lipophilicity of the extraction phase or reaction phase, respectively.

A purification process according to the present invention may, e.g., comprise liquid-liquid phase extraction of an L^{M}-containing species, whereas species that do not contain a moiety L^{M} essentially remain in the reaction mixture. It has to be understood that the term “essentially remains in the reaction mixture” as used in this context means that at least about 60%, preferably at least about 80% more preferably at least about 90% of each species that lacks a moiety L^{M} remains in the reaction mixture. Most preferably, at least about 99% or even 100% of each species containing no moiety L^{M} remain in the reaction mixture.

In embodiments of the present invention wherein the nucleophilic agent X is a radioisotope, preferably a radiohalogen such as ¹⁸F, it is desirable that the extraction medium and/or the moiety L^{M} do not contain a non-radioactive congener of X that may undergo an exchange reaction with the radioisotope. In accordance with this principle, it will also be understood that in cases where X comprises a radioisotope, it is desirable that the extraction medium and/or the moiety L^{M} do not contain a non-radioactive congener of said radioisotope. The absence of an extraction medium and/or a moiety L^{M} that does not contain a non-radioactive congener of X in a liquid-liquid extraction avoids yielding non-radioactive analogs of the species vector-X as by-products (e.g., “cold” vector-X).

As a non-limiting example, if X is ¹⁸F, it is desirable that L^{M} does not contain one or more ¹⁹F atoms which may undergo an exchange reaction with the radioisotope ¹⁸F. Such fluorine exchange reactions are well-known to those skilled in the art, and can, amongst other problems, lead to lower radiochemical yields and poor specific activity of the radiopharmaceutical.

In another preferred embodiment of the present invention, the liquid-liquid phase extraction process relies on the affinity of L^{M}-containing species to an extraction phase, such as a polar or ionic liquid extraction phase, whereas species that do not contain a moiety L^{M} are essentially not extractable into said liquid extraction phase, i.e. the embodiments are related to a liquid-liquid extraction process, wherein species that do not contain a moiety L^{M} are extracted and L^{M}-containing species essentially remain in the reaction mixture. Those of skill in the art will know how to generally perform a liquid-liquid extraction. A liquid-liquid extraction may be performed one, two, and in some cases even three, four, five or more times.

In yet another preferred embodiment, the liquid-liquid extraction process relies on the affinity of L^{M}-containing species to a liquid extraction phase that is not as polar as the reaction mixture, whereas a species that does not contain a moiety L^{M} is essentially not extractable into said liquid phase.

A “liquid extraction phase” as used in a liquid-liquid phase extraction method described herein is to be understood as a solution to which L^{M}-containing species are extracted, i.e. transferred to, from the reaction mixture, whereas a species that does not contain a purification moiety L^{M} essentially remains in the reaction mixture, i.e. is essentially non-extractable in said liquid extraction phase. In this context, essentially non-extractable means that at least about 60%, preferably at least about 85%, more preferably at least about 90% of each species containing no moiety L^{M} remain in the reaction mixture. Most preferably, at least about 99% or even 100% of each species containing no moiety L^{M} remain in the reaction mixture.

### Solid-Liquid Extraction

In another preferred embodiment of the present invention, the purification procedure comprises a solid-liquid phase extraction of L^{M}-containing species, whereas a species that does not contain a moiety L^{M} remains in the reaction mixture. A skilled person will generally know how to perform a solid-liquid extraction. Such an extraction may, e.g., comprise the use of beads which can be removed by centrifugation or filtration, or such an extraction may, e.g., comprise the use of columns and the like, wherein the solid phase is the stationary phase and the reaction mixture is or is present in the mobile phase. A resin can be a solid phase in accordance with the present invention. A resin can, e.g., be unmodified or can comprise one or more active and/or complementary groups attached to it. Preferably, a solid-liquid extraction according to the present invention relies on the affinity of a lipophilic L^{M} to a solid extraction phase. Alternatively, a solid-liquid extraction according to the present invention relies on a non covalent affinity between L^{M} and the solid extraction phase, wherein a combination of van der Waals, ionic and/or polar interactions is involved in the extraction process.

Furthermore, preferred embodiments of the present invention relate to processes wherein X is a radioisotope. In these cases a solid resin or a moiety attached thereon does not contain a non-radioactive congener of X that may undergo an exchange reaction with the radioisotope as explained above.

In accordance with this principle, it will also be understood when X comprises a radioisotope, it is desirable that the extraction medium and/or the leaving group L^{M} do not contain a non-radioactive congener of said radioisotope.

In yet another embodiment, the extraction process relies on a non-covalent affinity between L^{M} and a complementary compound bound to a resin, wherein a combination of van der Waals, ionic and/or polar interactions are involved.

### Purification by Precipitation

Following the teaching of the present invention, a L^{M}-containing species can be separated from a species that does not contain a moiety L^{M} by means of precipitating a L^{M}-containing species, i.e., the purification procedure comprises adjusting the reaction mixture to conditions so that the L^{M}-containing species precipitate whereas a species that does not contain a moiety L^{M} remains soluble.

The precipitation of a L^{M}-containing species can be achieved by, e.g., adjusting the polarity, the pH, the temperature and/or the ion strength of the reaction mixture and/or by adding, e.g., specific ions to the reaction mixture so that L^{M}-containing species precipitate whereas a species that does not contain a moiety L^{M} remains soluble. For example, L^{M} can comprise cholesteryl, which are prone to precipitate when added to water. Such precipitated species can easily be removed by filtration or centrifugation.

### Kits

Another aspect of the present invention relates to kits for carrying out a nucleophilic substitution reaction and/or purification procedures as described herein.

According to some embodiments of the present invention, the kit may contain the compounds L^{M} and vector-L^{M} in any suitable combination, and additionally may optionally comprise a species X or a precursor of X such as X* or X**. In particular, a radioactive species X may be supplied with the kit if it has a suitably long half life to accommodate, manufacture, release, shipping, and receipt of the kit, but it also may be omitted if radioactive X has to be produced at the site of use (e.g. by a cyclotron).

Sometimes, the kit may furthermore comprise a product manual mentioning one or more suitable vector-L^{M} or vector moieties, respectively, or counterparts to synthesize vector-L^{M} and reaction conditions to perform said synthesis. Optionally, the product manual may describe one or more experimental protocols how to perform a synthesis of vector-L^{M}, and/or one or more experimental protocols how to perform a nucleophilic substitution reaction according to the present invention (i.e. one or more experimental protocols of how to perform a synthesis of vector-X or vector-X′, respectively), and/or one or more experimental protocols to perform a purification of vector-X.

In addition, a kit for carrying out a process in accordance with embodiments of the present invention may further comprise a nucleophilic agent X or a precursor of X as described herein.

It will be understood that the compound(s) included in the kit is/are delivered as part of a single reaction mixture, or separately packaged into one or a plurality of suitable containers. It is in some instances advantageous if the kit will further comprise a liquid-soluble support and/or a suitable reaction medium.

The kits of the present invention may further comprise a liquid extraction phase or the compounds to prepare a liquid extraction phase for separating L^{M}-containing species from a species that does not contain a moiety L^{M} by a purification procedure as described herein.

Optionally, the kit may further comprise at least one extraction resin to separate L^{M}-containing species from a species that does not contain a moiety L^{M} as described herein and/or the kit may comprise compounds to achieve conditions within the reaction mixture so that the L^{M}-containing species precipitate whereas a species that does not contain a purification moiety L^{M} remains soluble. Such compounds may be acids or bases to adjust the pH, organic solvents to adjust the polarity or salts to adjust the ion strength of the reaction mixture.

In another embodiments, the kit will also include a resin comprising a complementary reactive group which is suitable to covalently bind to a reactive group on the moiety L^{M} of an L^{M}-containing species so as to separate L^{M}-containing species from a species that does not contain a purification moiety.

The kits of the present invention may comprise the various compounds or media as one or more solutions that are in ready to use form (i.e., all components are present in the desired concentration to carry out a method according to the present invention), or they may contain one or several compounds or media in the form of a concentrated solution that is to be diluted with a pre-determined amount of solvent prior to their use. The concentration of such a stock solution may be, without limiting the scope, 1.5×, 2×, 2.5×, 5×, 10×, 50×, 100×, or 1000× of the concentration of a ready to use solution. Alternatively, the kit may comprise one or several compounds or media in dry form or lyophilized form that are to be dissolved with a suitable solvent to the appropriate concentration for use in a method according to the present invention

It will be understood that all of the preferred compounds described herein, as well as the preferred media and embodiments to purify a species lacking a moiety L^{M} may be included in the kits of the present invention.

It is generally preferred that each component, each dried component, each stock solution or solution ready to use (such as the reaction medium or a liquid extraction phase) will be separately placed in a sealed container, although it will be apparent to those of skill in the art that other combinations and packaging options may be possible and useful in certain situations. For example, the precursor vector-L^{M} may already be combined with the reaction mixture.

It will be apparent to those of skill in the art that many modifications and variations of the embodiments described herein are possible without departing from the spirit and scope of the present invention. The present invention and its advantages are further illustrated in the following, non-limiting examples.

### EXAMPLES

### Example 1

### Synthesis of a Non-Polar Leaving Group: Cesyl Chloride (1)

A schematic overview of the synthesis of the non-polar leaving group; (1) as a radiolabelling precursor is given in Scheme 1 below.

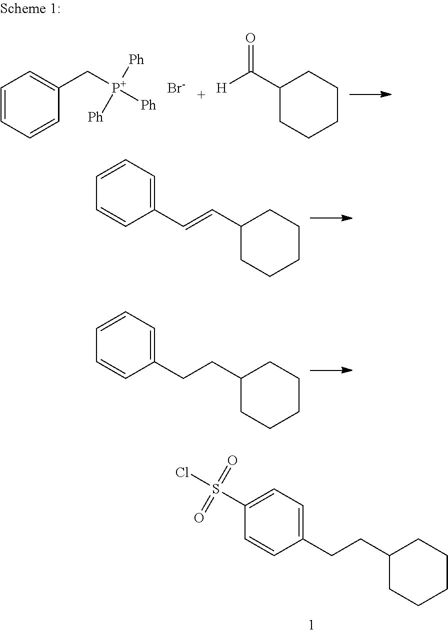

### Synthesis of ((E)-2-Cyclohexyl-vinyl)-benzene

To a solution of benzyltriphenylphosphonium bromide (1.0 g, 2.06 mmol) in dry THF (20 mL) at 0° C. was slowly added LDA (1.5 mL, 3.1 mmol, 2 M solution in THF) solution under nitrogen atmosphere. After 30 min, cyclohexane carboxaldehyde (0.31 g, 2.7 mmol) in THF (3 mL) was added over 10 min. After stirring for 1 h at 0° C., the reaction mixture was allowed to warm to room temperature. After stirring overnight at room temperature, the reaction mixture was quenched with saturated aqueous NH4Cl solution and extracted with ethyl acetate (3×20 mL). The combined organic layer was washed with brine, and dried over Na2SO4 and concentrated under reduced pressure. The crude product was passed through a short silica gel column bed by eluting hexane to give the product (0.39 g, 91%) as a colorless liquid.

### Synthesis of (2-Cyclohexyl-ethyl)-benzene

To a solution of ((E)-2-cyclohexyl-vinyl)-benzene (1.0 g, 5.37 mmol) in ethyl acetate (15 mL) was added palladium on charcoal (10% Pd/C, 20 mg). The flask was then connected to a hydrogen balloon. The suspension was carefully degassed, and recharged with hydrogen. After stirring for 8 h at room temperature, the Pd/C catalyst was removed by filtration with a Celite pad and the resulting filtrate were concentrated by a rotary evaporator. Crude product was passed through short silica gel column by eluting n-hexane to obtain (2-Cyclohexyl-ethyl)-benzene (0.98 g, 97%) as a colorless liquid.

### Synthesis of 4-(2-Cyclohexyl-ethyl)-benzenesulfonyl chloride (1)-Cesyl Chloride

To a mixture of chlorosulfonic acid (2.9 mL, 43.0 mmol) and NaCl (70 mg, 15.2 mmol) in CHCl3 (20 mL) was added a solution of (2-Cyclohexyl-ethyl)-benzene (1.34 g, 7.1 mmol) in CHCl3 (5 mL) at 0° C. After stirring for 2 h at room temperature, the reaction mixture was cautiously poured into crushed ice water (100 mL) and successively extracted with CH₂Cl₂. The combined extracts were washed with 10% NaHCO3, brine, and dried over Na2SO4, and concentrated by a rotary evaporator. Silica gel column chromatography of crude product was performed eluting n-hexane to give 1 (1.63 g, 80%) as a colorless liquid.

### Example 2

### Synthesis of a Non-Polar Leaving Group: Dipsyl Chloride (2)

A schematic overview of the synthesis of the non-polar leaving group; (2) as a radiolabelling precursor is given in Scheme 1 below.

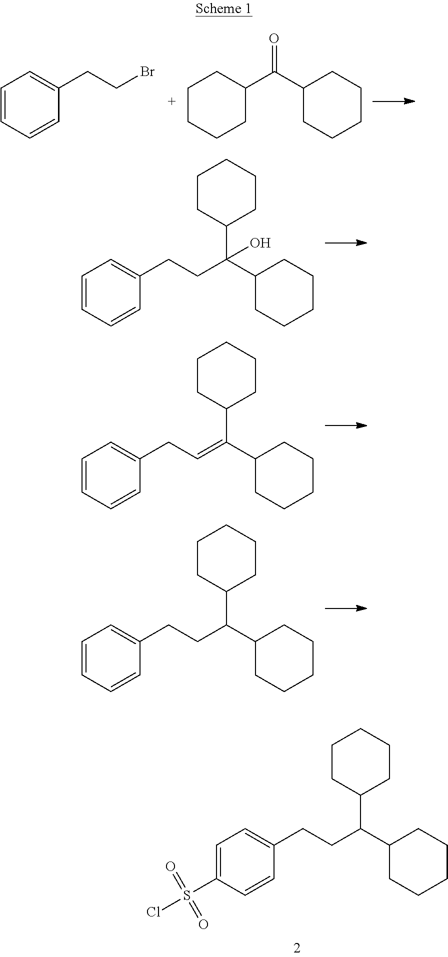

### Synthesis of 1,1-Dicyclohexyl-3-phenyl-propan-1-ol

To a THF (25 mL) solution containing Mg tuning (0.5 g, 20.5 mmol) were added 2-bromophenyl ethane (2.55 mL, 18 mmol) at −20° C. under N2. After 1 h, a solution of dicylohexyl ketone (3.7 mL 18 mmol) in THF (15 mL) was added dropwise into the solution over 15 min. After stirring for 3 h at room temperature, the reaction was quenched with 1 M aqueous HCl, and the reaction mixture was then filtered through a Celite pad, and the filtrate was extracted with ethyl acetate (3 l 30 mL), the combined organic layer was washed with brine, dried over Na2SO4, and concentrated by a rotary evaporator. The crude product was recrystallized with ethyl acetate and hexane to obtain 1,1-dicyclohexyl-3-phenyl-propan-1-ol (4.7 g, 84%) as a white solid.

### Synthesis of (3,3-Dicyclohexyl-allyl)-benzene

1,1-Dicyclohexyl-3-phenylpropanol (6, 4.0 g, 13.3 mmol) was dissolved in dichloromethane (40 mL) and triethylamine (9.3 mL, 66.6 mmol) under −20° C. After 10 min, a solution of methanesulfonyl chloride (1.1 mL, 14.6 mmol) in dichloromethane (2 mL) was added. After 10 min, the reaction mixture was allowed to warm to room temperature, and stirred for 6 h. The reaction mixture was concentrated by a rotary evaporator, and the crude product was filtered through a short silica gel bed eluting n-hexane to afford (3,3-dicyclohexyl-allyl)-benzene (3.3 g, 89%) as a colorless syrup.

### Synthesis of (3,3-Dicyclohexyl-propyl)-benzene

To a solution of (3,3-dicyclohexyl-allyl)-benzene (1.0 g, 3.54 mmol) in ethyl acetate (15 mL) was added palladium on charcoal (10% Pd/C, 20 mg). The flask was then connected to a hydrogen balloon. The suspension was carefully degassed, recharged with hydrogen. After stirring for 8 h at room temperature, the Pd/C catalyst was removed by filtration with a Celite pad, and the filtrate was evaporated by a rotary evaporator. The crude product was passed through a short silica gel column by eluting n-hexane to obtain (3,3-dicyclohexyl-propyl)-benzene (990 mg, 99%) as a colorless liquid.

### Synthesis of 4-(3,3-Dicyclohexyl-propyl)-benzenesulfonyl chloride (2)-Dipsyl Chloride

To a mixture of chlorosulfonic acid (1.4 mL, 21.0 mmol) and NaCl (32 mg, 7 mmol) in CHCl3 (20 mL) was added a solution of (3,3-dicyclohexyl-propyl)-benzene (1.0 g, 3.5 mmol) in CHCl3 (5 mL) at 0° C. After stirring for 2 h at room temperature, the reaction mixture was cautiously poured into crushed ice water (100 mL) and extracted with CH₂Cl₂. The combined extracts were washed with 10% NaHCO3, brine, and dried over Na2SO4 and concentrated by a rotary evaporator. Silica gel column chromatography of crude product was performed eluting n-hexane to gave Dipsyl chloride 2 (1.0 g, 80%) as a white solid.

### Example 3

### Synthesis of a Non-Polar Leaving Group: Cholesyl Chloride (3)

A schematic overview of the synthesis of the non-polar leaving group; (3) as a radiolabelling precursor is given in Scheme 3 below.

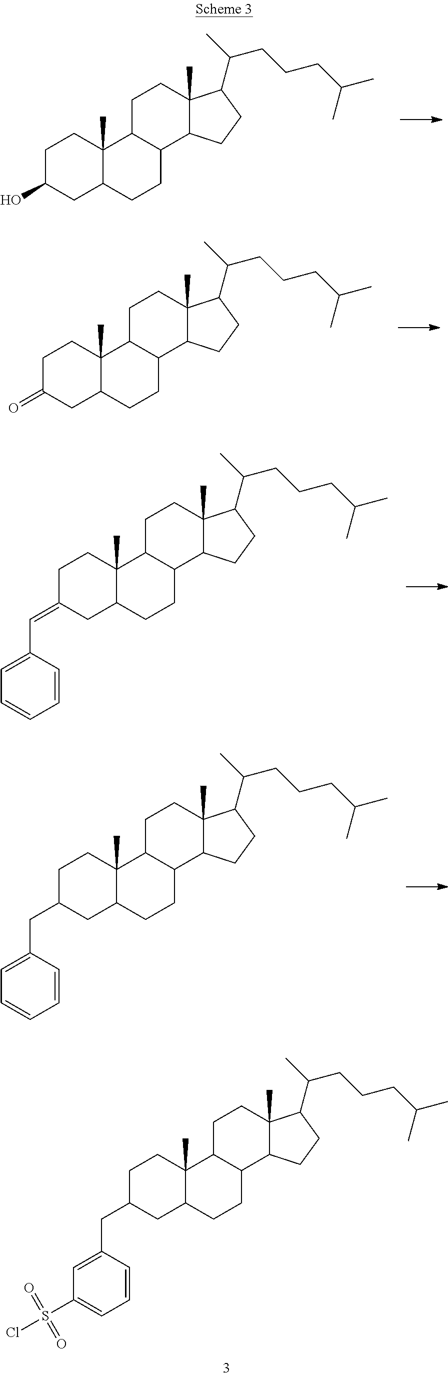

### Synthesis of (10S,13R)-17-(1,5-Dimethyl-hexyl)-10,13-dimethyl-hexadecahydrocyclopenta[a]phenanthren-3-one

Cholesterol (5.0 g, 12 mmol) was dissolved in acetone (50 mL) at 0° C., and titrated with 8 N Jones reagent after 2 h of vigorous stirring at 0° C. The reaction mixture was poured into a cold half saturated NaCl solution and extracted with ethyl acetate (30 mL×3). The ethyl acetate layer was repeatedly washed with 5% NaHCO₃ and dried over Na₂SO₄, and concentrated under vacuum to provide (10S,13R)-17-(1,5-dimethyl-hexyl)-10,13-dimethyl-hexadecahydrocyclopenta[a]phenanthren-3-one (3.77 g, 76%) as white solid.

### Synthesis of (10S,13R)-17-(1,5-Dimethyl-hexyl)-10,13-dimethyl-3-[1-phenyl-meth-(Z)-ylidene]-hexadecahydro-cyclopenta[a]phenanthrene

To a solution of benzyltriphenylphosphonium bromide (1.0 g, 2.0 mmol) in dry THF (20 mL) was slowly added LDA (2 M solution in THF, 1.5 mL, 3.1 mmol) at 0° C. under nitrogen. After 30 min, (10S,13R)-17-(1,5-dimethyl-hexyl)-10,13-dimethyl-hexadecahydrocyclopenta[a]phenanthren-3-one (0.73 g, 1.8 mmol) in THF (3 mL) was added into the solution over 10 min, and then the reaction mixture was allowed to warm to room temperature. After refluxing for 3 h, the reaction mixture was diluted with water and extracted with ethyl acetate (3×20 mL). The combined organic layers were washed with brine, and dried over Na₂SO₄ and concentrated by a rotary evaporator. The crude product was passed through the short silica gel column bed by eluting n-hexane to give (10S,13R)-17-(1,5-Dimethyl-hexyl)-10,13-dimethyl-3-[1-phenyl-meth-(Z)-ylidene]-hexadecahydro-cyclopenta[a]phenanthrene (0.71 mg, 82%) as a white solid.

### Synthesis of (10S,13R)-3-Benzyl-17-(1,5-dimethyl-hexyl)-10,13-dimethyl-hexadecahydro-cyclopenta[a]phenanthrene

To a solution of (10S,13R)-17-(1,5-Dimethyl-hexyl)-10,13-dimethyl-3-[1-phenyl-meth-(Z)-ylidene]-hexadecahydro-cyclopenta[a]phenanthrene (1.0 g, 2.17 mmol) in ethyl acetate (15 mL) was added palladium on charcoal (10% Pd/C, 20 mg). The flask was then connected to a hydrogen balloon. The suspension was carefully degassed, and recharged with hydrogen gas. After stirring for 8 h at room temperature, the Pd/C catalyst was removed by filtration with a Celite pad and the filtrate were concentrated by a rotary evaporator. The crude product was passed through a short silica gel column by eluting n-hexane to obtain (10S,13R)-3-benzyl-17-(1,5-dimethyl-hexyl)-10,13-dimethyl-hexadecahydro-cyclopenta[a]phenanthrene (0.99 g, 99%).

### Synthesis of 3-[(10S,13R)-17-(1,5-Dimethyl-hexyl)-10,13-dimethyl-hexadecahydro-cyclopenta[a]phenanthren-3-ylmethyl]-benzenesulfonyl chloride (3)-Cholesyl Chloride

To a mixture of chlorosulfonic acid (0.89 mL, 13.0 mmol) and NaCl (20 mg, 4.3 mmol) in CHCl₃ (20 mL) was added a solution of (10S,13R)-3-benzyl-17-(1,5-dimethyl-hexyl)-10,13-dimethyl-hexadecahydro-cyclopenta[a]phenanthrene (1.0 g, 2.1 mmol) in CHCl₃ (5 mL) at 0° C. After stirring for 1 h at room temperature, the reaction mixture was cautiously poured into crushed ice water (100 mL) and successively extracted with CH₂Cl₂. The combined extracts were washed with 10% NaHCO₃, brine, and dried over Na₂SO₄, and concentrated by a rotary evaporator. Silica gel column chromatography of crude product was performed eluting n-hexane to give Cholesyl chloride 3 (750 mg, 62%) as a white solid.

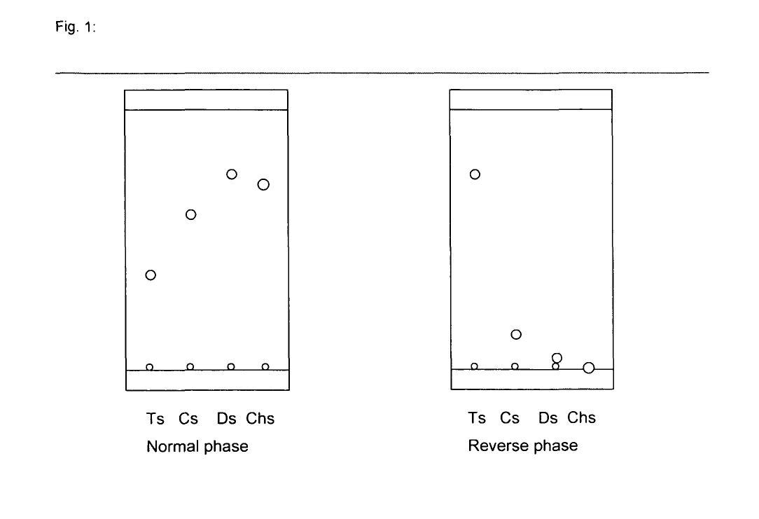

See FIG. 1 for thin Layer Chromatography (TLC) of non-polar leaving groups versus Tosyl chloride (polar).

FIG 1. TLC of four different sulfonyl chlorides
in normal phase and reverse phase.
Silica gel TLC (normal phase system)
Elute solution = 3:97 (EtOAc:hexane)
TLC analysis left to right:

| | |
|---|---|
| Tosyl chloride | ( ) Rf = 0.41 |
| Cesyl chloride (1) | ( ) Rf = 0.61 |
| Dicpsyl chloride (2) | ( ) Rf = 0.79 |
| Cholsyl chloride (3) | ( ) Rf. = 0.71 |

C-18 TLC (reverse phase system)
Elute solution = 5:5 (MeOH:H₂O)
TLC analysis left to right:

| | |
|---|---|
| Tosyl chloride | ( ) Rf = 0.70 |
| Cesyl chloride (1) | ( ) Rf = 0.21 |
| Dicpsyl chloride (2) | ( ) Rf = 0.06 |
| Cholsyl chloride (3) | ( ) Rf = 0.00 |

Ts = Tosyl Chloride;
Cs = Cesyl Chloride (6);
Ds = Dipsyl Chloride (7);
Chs = Cholesyl Chloride (8)

### Example 4

### Synthesis of a FDDNP Precursor with the Cesyl Non-Polar Leaving Group: (4)

A schematic overview of the synthesis of the non-polar leaving group; (9) as a radiolabelling precursor is given in Scheme 4 below.

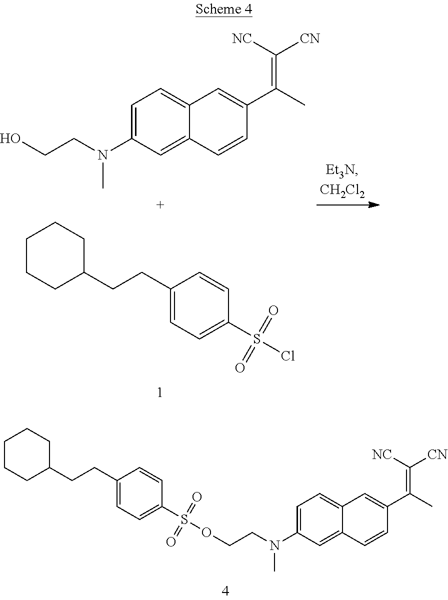

### Synthesis of 2-(1,1-Dicyanopropen-2-yl)-6-(2-(4-(2-cyclohexylethyl)benzenesulfonyl oxyethyl)methylamino naphthalene (4)

2-(1,1-Dicyanopropen-2-yl)-6-(2-hydroxyethyl)-methylamino-naphthalene (100 mg, 0.34 mmol) was dissolved in anhydrous pyridine (5 mL) and Cesyl chloride (1, 324 mg, 1.13 mmol) was added into the solution. After stirring for 4 h at room temperature, the reaction mixture was diluted with ethyl acetate, and then washed with H₂O, 1N HCl, and aqueous NaHCO₃. The organic layer was dried over Na₂SO₄ and evaporated to dryness in vacuo. Purification by flash column chromatography (30% ethyl acetate/hexane) afforded the product 4 (125 mg, 68%) as a reddish foamy solid.

### Example 5

### Radiosyntheses of FDDNP from the Cesyl Precursor (Non-Polar)

In radiofluorination [¹⁸F]Fluoride (185 MBq) was eluted from a QMA cartridge (equilibrated with 0.5 M K₂CO₃, washed with 10 ml H2O) with 0.6 mL of 1/1 H₂O/acetonitrile containing 22 mg Kryptofix© (K222) and 7 mg K₂CO₃ into a reaction vial. The solvents were evaporated and the residue dried at 100° C. under a light N₂-stream, more acetonitrile was added, and the drying process was repeated. Precursor (4) (4 mg) in 500 μL MeCN was added to the reaction vial, the reaction stirred for 10 min at 100° C. The reaction mixture was analyzed by radioTLC and HPLC. For results see Table 2. HPLC conditions: C-8 reversed phase column acetonitrile/water=65/35, flow=4 ml/min.

### Example 6

### Synthesis of a FLT Precursor with the Cesyl Non-Polar Leaving Group: (5)

A schematic overview of the synthesis of the non-polar leaving group; (5) as a radiolabelling precursor is given in Scheme 9 below.

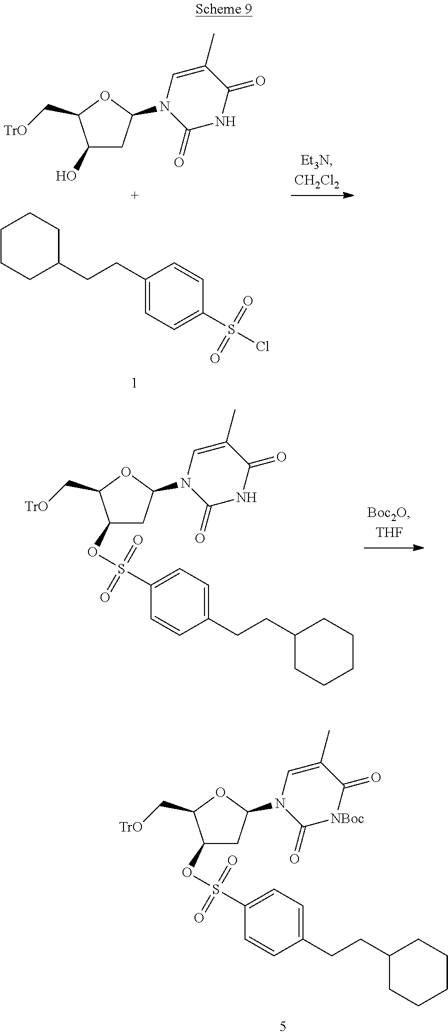

### Synthesis of [5′-O-triphenylmethyl-T-deoxy-3′-O-(4-(2-cyclohexylethyl)benzenesulfonyl)-β-D-threopentofuranosyl]thymine

1-[5′-O-Triphenylmethyl-2′-deoxy-β-D-threopentofuranosyl]thymine (0.93 g, 1.91 mmol) was dissolved in anhydrous pyridine (10 mL) and the solution was cooled to 0° C. Cesyl chloride 1 (1.08 g, 3.75 mmol) and silver trifluoromethane sulfonate (0.96 g, 3.75 mmol) were added. The reaction mixture was stirred for 50 min at 0° C., and for 2 h at room temperature. The reaction was quenched with ethyl acetate (50 mL). The resultant precipitation was filtered. The reaction mixture was washed with brine (20 mL). The organic layer was dried over Na₂SO₄ and evaporated to dryness in vacuo. Purification by flash column chromatography (60% ethyl acetate/hexane) afforded the product [5′-O-triphenylmethyl-2′-deoxy-3′-O-(4-(2-cyclohexylethyl)benzenesulfonyl)-β-D-threopentofuranosyl]thymine (1.1 g, 78%) as a yellowish foamy solid.

### Synthesis of 3-N-t-butoxycarbonyl-[5′-O-triphenylmethyl-2′-deoxy-3′-O-(4-((N-methylmethylsulfonamido)ethyl)benzenesulfonyl)-β-D-threopentofuranosyl]thymine (5)

[5′-O-triphenylmethyl-2′-deoxy-3′-O-(4-((N-methylmethylsulfonamido)ethyl)benzenesulfonyl)-β-D-threopentofuranosyl]thymine. (0.3 g, 0.40 mmol) was dissolved in THF (10 mL) and t-butoxycarbonyl anhydride (0.094 g, 0.434 mmol) was added. After stirring for 80 min at room temperature, a stoichiometric amount of dimethylaminopyridine (DMAP) was added, and stirring was continued for 4 h at room temperature. The reaction mixture was diluted with ethyl acetate, and washed with H2O, 1N HCl, and aqueous NaHCO3. The organic layer was dried over Na2SO4 and evaporated in vacuo. Purification by flash column chromatography (50% ethyl acetate/methylene chloride) afforded the product 5 (0.17 g, 50%) as a yellowish foamy solid.

### Example 6

### Radiosyntheses of FLT from the Cesyl Precursor 5 (Non-Polar)

In radiofluorination [¹⁸]-Fluoride (185 MBq) was eluted from a Chromafix cartridge (equilibrated with 10 ml H2O) with 0.6 mL of 1/1 H₂O/acetonitrile containing 10 μl TBAHCO3 into a reaction vial. The solvents were evaporated and the residue dried at 100° C. under a light N₂-stream, more acetonitrile was added, and the drying process was repeated. Precursor (5) (20 mg) in 100 μL MeCN and 500 ml tBuOH was added to the reaction vial, the reaction stirred for 15 min at 120° C. The reaction mixture was analyzed by radioTLC and HPLC. For results see Table 3. HPLC conditions: C-8 reversed phase column, methanol/water=75/25, flow=3 ml/min.

### 1) RadioTLC

[¹⁸F]fluorination yield in [¹⁸F]FLT synthesis by RadioTLC (%)

| Leaving group | [¹⁸F]fluorination yield in radioTLC analysis |
|---|---|
| ONs | 91.68 |
| OCs (5) | 77.81 |

^{a}All reactions were carried out using the same reaction conditions. 20 mg precursor, 10 μl TBAHCO3, 0.5 ml tBuOH and 0.1 ml MeCN at 120°0 for 15 min.

### Example 7

### Comparison of Leaving Groups for FDDNP

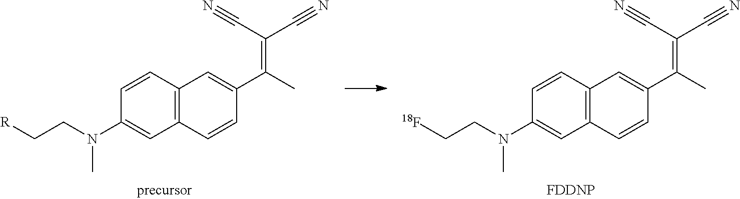

| | | | Difference |
|---|---|---|---|
| | | | lopD(FDDNP) |
| | | | and |
| R = | | logD | logD(precursor) |
| F | | 3.24 | |
| OMs | 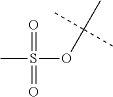 | 2.33 | 0.91 |
| OTs | 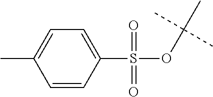 | 3.47 | 0.23 |
| OTf | 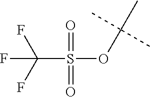 | 3.09 | 0.15 |
| ONs | 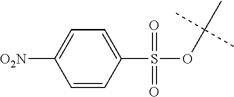 | 2.96 | 0.28 |
| OCs | 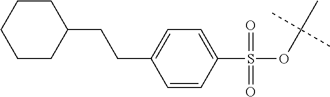 | 5.48 | 2.24 |
| ODs | 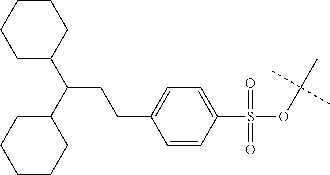 | 7.99 | 4.75 |
| OChs | 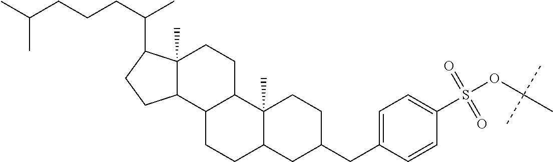 | 12.61 | 9.37 |

Differences of logD values of 2.24 for Cesyl-precursor, 4.75 for Dipsyl-precursor and 9.37 for Cholesyl-precursor support of purification by solid-phase extraction in contrast to max. 0.91 difference in logD for commonly used precursors.

### Example 8

### Comparison of Leaving Groups for THP-FMISO

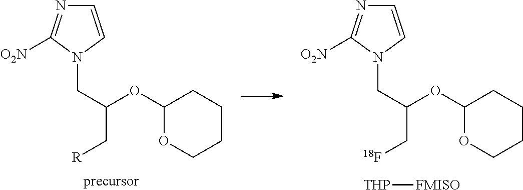

| | | | Difference |
|---|---|---|---|
| | | | lopD(THP—FMISO) |
| | | | and |
| R = | | logD | logD(precursor) |
| F | | 1.77 | |
| OMs | 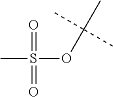 | 0.93 | 0.84 |
| OTs | 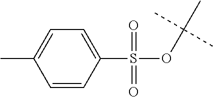 | 2.07 | 0.30 |
| OTf | 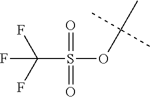 | 1.70 | 0.07 |
| ONs | 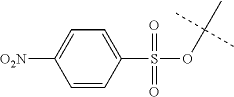 | 1.56 | 0.21 |
| OCs | 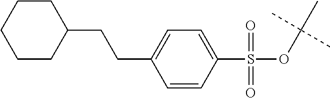 | 4.08 | 2.31 |
| ODs | 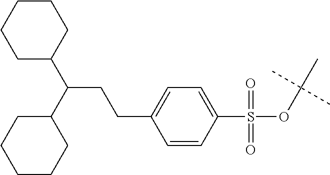 | 6.60 | 4.83 |
| OChs | 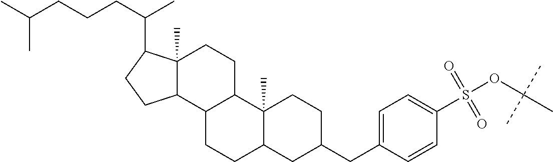 | 11.22 | 9.45 |

Differences of logD values of 2.31 for Cesyl-precursor, 4.83 for Dipsyl-precursor and 9.45 for Cholesyl-precursor support of purification by solid-phase extraction in contrast to max. 0.84 difference in logD for commonly used precursors.

### Example 9

### Comparison of Leaving Groups for FET (Protected)

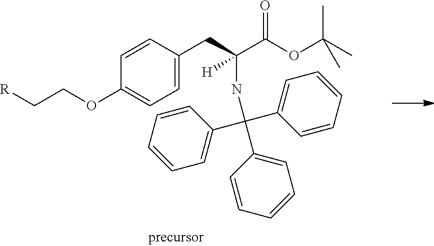

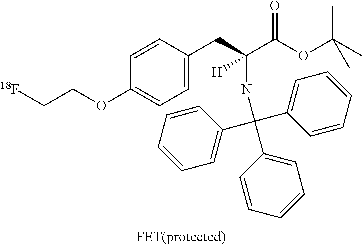

| | | | Difference |
|---|---|---|---|
| | | | lopD(FET(protected)) |
| | | | and |
| R = | | logD | logD(precursor) |
| F | | 7.67 | |
| OMs | 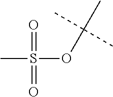 | 6.67 | 1.00 |
| OTs | 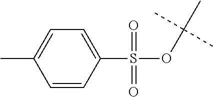 | 7.81 | 0.14 |
| OTf | 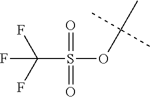 | 7.43 | 0.24 |
| ONs | 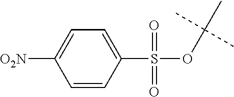 | 7.30 | 0.37 |
| OCs | 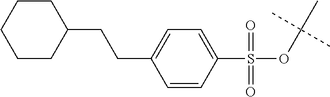 | 9.82 | 2.15 |
| ODs | 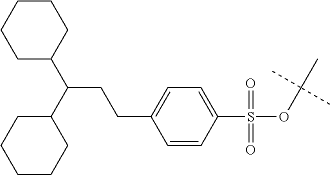 | 12.34 | 4.67 |
| OChs | 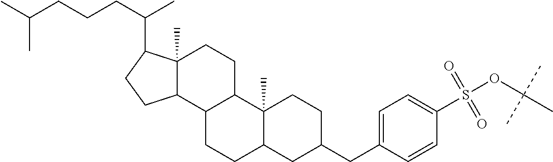 | 16.95 | 9.28 |

Differences of logD values of 2.15 for Cesyl-precursor, 4.67 for Dipsyl-precursor and 9.28 for Cholesyl-precursor support of purification by solid-phase extraction in contrast to max. 1.00 difference in logD for commonly used precursors.

### Example 10

### Comparison of Leaving Groups for MMTr-Boc-FLT

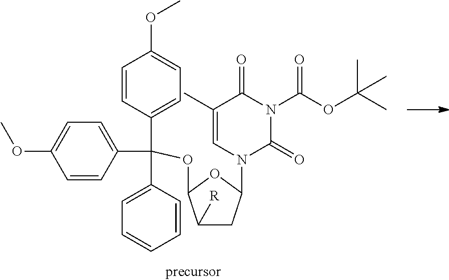

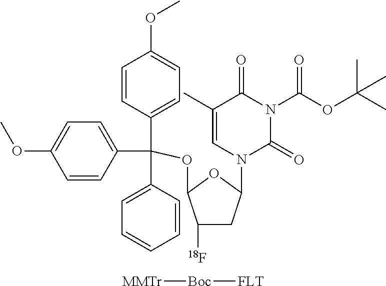

| | | | Difference lopD |
|---|---|---|---|
| | | | (MMTr—Boc—FLT) |
| R = | | | and logD(precursor) |
| F | | 4.76 | |
| OMs | 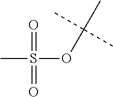 | 4.06 | 0.70 |
| OTs | 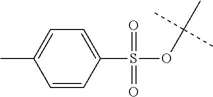 | 5.16 | 0.40 |
| OTf | 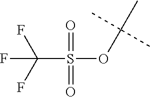 | 4.82 | 0.06 |
| ONs | 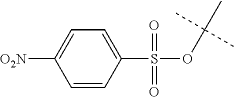 | 4.65 | 0.11 |
| OCs | 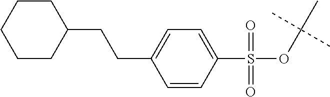 | 7.17 | 2.41 |
| ODs | 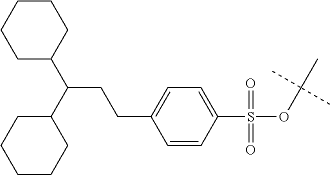 | 9.68 | 4.92 |
| OChs | 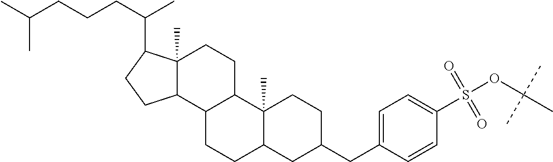 | 14.30 | 9.54 |

Differences of logD values of 2.41 for Cesyl-precursor, 4.92 for Dipsyl-precursor and 9.54 for Cholesyl-precursor support of purification by solid-phase extraction in contrast to max. 0.70 difference in logD for commonly used precursors.

### Example 11

### Comparison of Leaving Groups for Boc-Pystilbene1

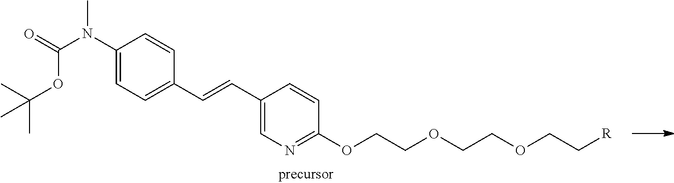

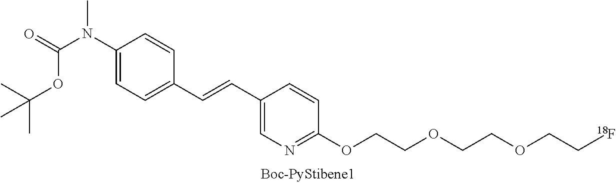

| | | | Difference |
|---|---|---|---|
| | | | lopD(Boc-PyStilbene1) |
| R = | | logD | and logD(precursor) |
| F | | 4.11 | |
| OMs | 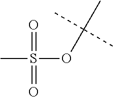 | 3.04 | 1.07 |
| OTs | 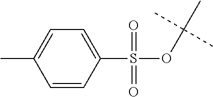 | 4.18 | 0.07 |
| OTf | 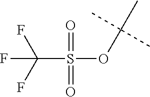 | 3.80 | 0.31 |
| ONs | 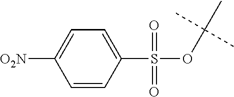 | 3.67 | 0.44 |
| OCs | 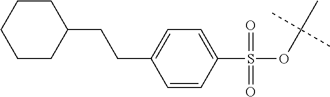 | 6.19 | 2.08 |
| ODs | 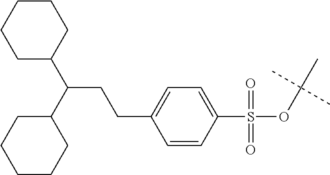 | 8.71 | 4.60 |
| OChs | 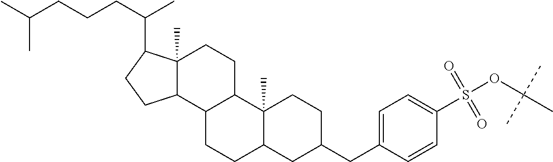 | 13.32 | 9.21 |

Differences of logD values of 2.08 for Cesyl-precursor, 4.60 for Dipsyl-precursor and 9.21 for Cholesyl-precursor support of purification by solid-phase extraction in contrast to max. 1.07 difference in logD for commonly used precursors.

### Example 12

### Comparison of Leaving Groups for BMS747

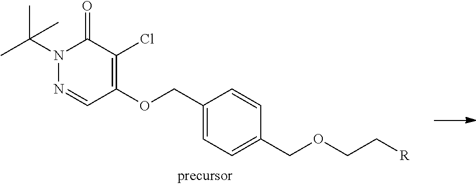

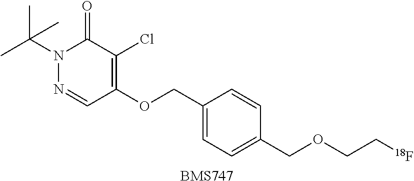

| | | | Difference |
|---|---|---|---|
| | | | lopD(BMS747) and |
| R = | | logD | logD(precursor) |
| F | | 2.93 | |
| OMs | 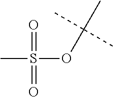 | 1.86 | 1.07 |
| OTs | 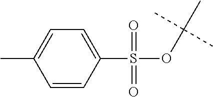 | 2.99 | 0.06 |
| OTf | 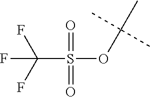 | 2.62 | 0.31 |
| ONs | 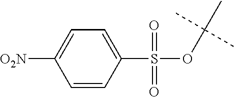 | 2.49 | 0.44 |
| OCs | 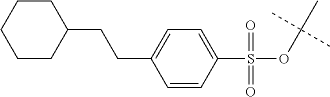 | 5.00 | 2.07 |
| ODs | 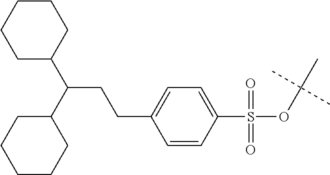 | 7.52 | 4.59 |
| OChs | 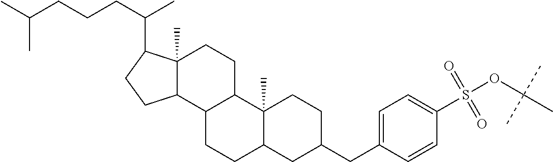 | 12.14 | 9.21 |

Differences of logD values of 2.07 for Cesyl-precursor, 4.59 for Dipsyl-precursor and 9.21 for Cholesyl-precursor support of purification by solid-phase extraction in contrast to max. 1.07 difference in logD for commonly used precursors.

### Example 13

### Comparison of Leaving Groups for FP-CIT

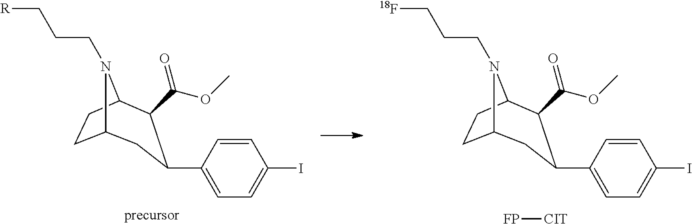

| | | | Difference |
|---|---|---|---|
| | | | lopD(FP—CIT) |
| | | | and |
| R = | | logD | logD(precursor) |
| F | | 3.76 | |
| OMs | 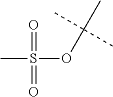 | 2.83 | 0.93 |
| OTs | 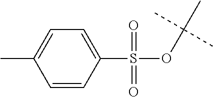 | 3.97 | 0.21 |
| OTf | 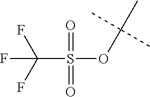 | 3.60 | 0.16 |
| ONs | 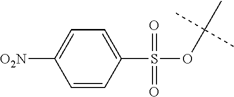 | 3.46 | 0.30 |
| OCs | 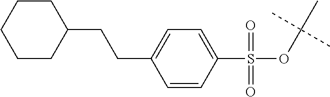 | 5.98 | 2.22 |
| ODs | 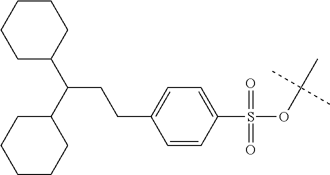 | 8.50 | 4.74 |
| OChs | 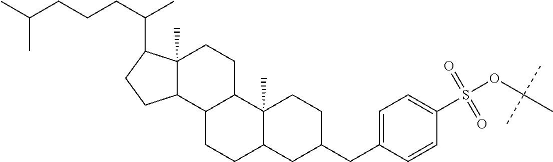 | 13.12 | 9.36 |

Differences of logD values of 2.22 for Cesyl-precursor, 4.74 for Dipsyl-precursor and 9.36 for Cholesyl-precursor support of purification by solid-phase extraction in contrast to max. 0.93 difference in logD for commonly used precursors.

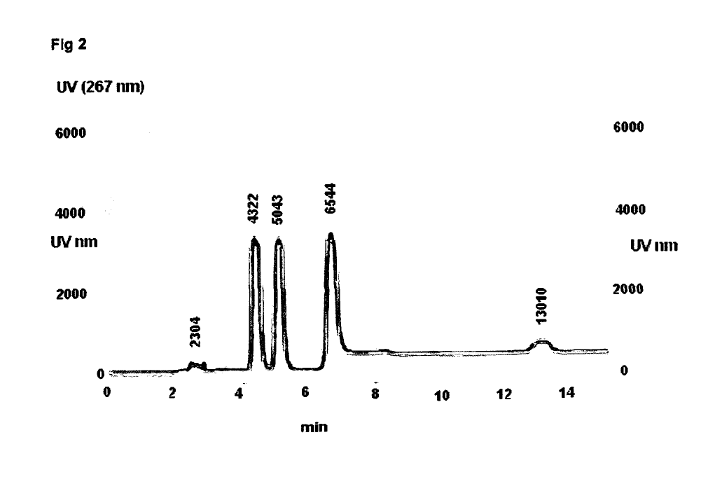

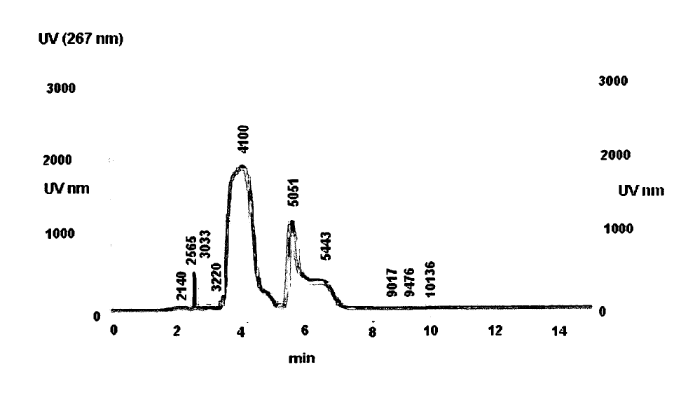

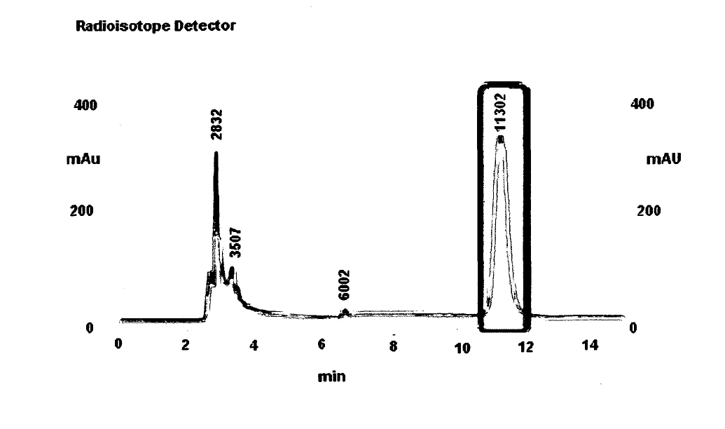

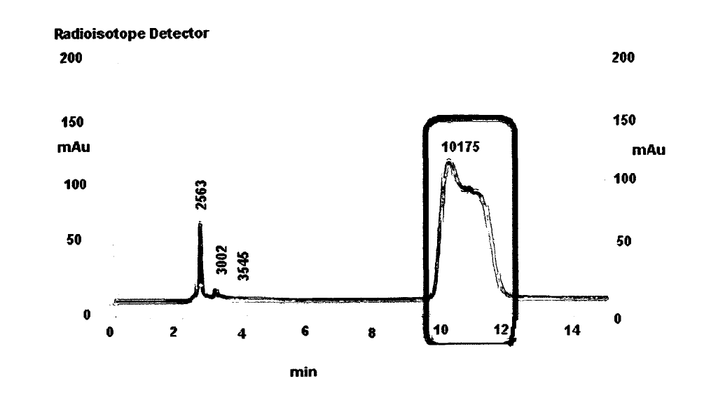

FIG. 2 shows Nosylate leaving group showed big organic impurities peak at before & after true [¹⁸F]FLT peak. Due to these organic impurities peaks form the nosylate, there is a high possibility of contamination in the final product, therefore, HPLC purification methods are mandatory. However the Cs leaving group showed less impurities, these impurities were all more polar and did not elute near the product peak. Therefore, solid phase extraction (SPE) methods could be used instead of HPLC methods, making the process simpler and more efficient.

1. A method of preparation of compound of Formula II by direct nucleophilic radiofluorination of compound of Formula I 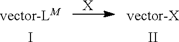
wherein
the difference between the logD of compound of Formula I and the logD of compound of Formula II is greater than 1.5,
the vector is a targeting vector, and
L^{M} is a modified leaving group, suitable for direct nucleophilic fluorination,
X is a nucleophilic moiety, preferably ¹⁸F.

2. The method according to claim 1, wherein difference between the logD of compound of Formula I and the logD of compound of Formula II is greater than 2, more preferably greater than 4

3. The method of claim 1, wherein L^{M} is a sulfonate derivative.

4. The method of claim 1, wherein compound of Formula I is selected from the group comprising

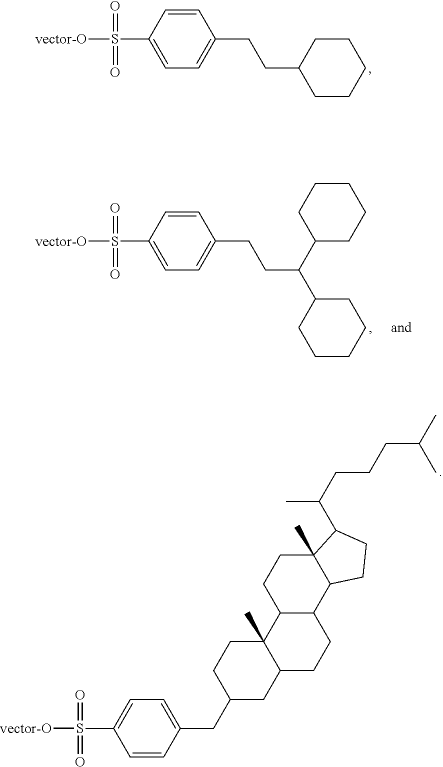

5. A method for separating a compound of Formula II from compound of Formula I and side products resulting from a nucleophilic substitution reaction according to claim 1.

6. The method according to claim 5, wherein compound of Formula II is separated from compound of Formula I by solid-phase-extraction, filtration, precipitation, distillation or liquid-liquid-extraction.

7. A compound of Formula I:
vector-L^{M} I
that is a precursor for a direct nucleophilic radiofluorination compound of Formula II:
vector-X II
wherein
the difference between the logD of compound of Formula I and the logD of compound of Formula II is greater than 1.5, and
vector is a targeting vector,
X is a nucleophilic moiety preferably ¹⁸F and
L^{M} is a modified leaving group suitable for direct nucleophilic fluorination.

8. The compound of claim 7, wherein difference of logD of compound of Formula I and the logD of compound of Formula II is greater than 2, more preferably greater than 4.

9. The compound according to claim 7 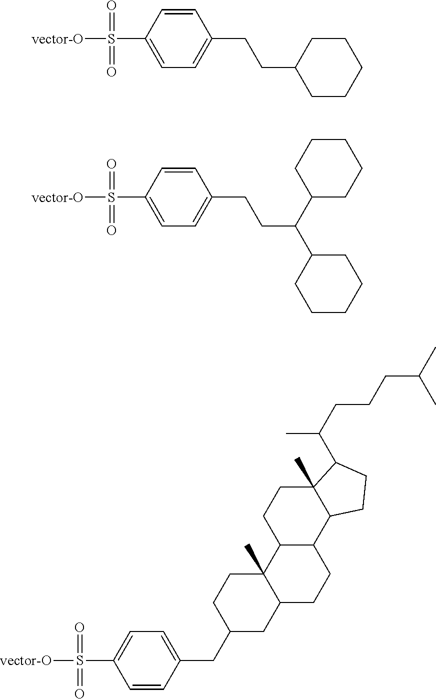
wherein vector is a targeting vector; 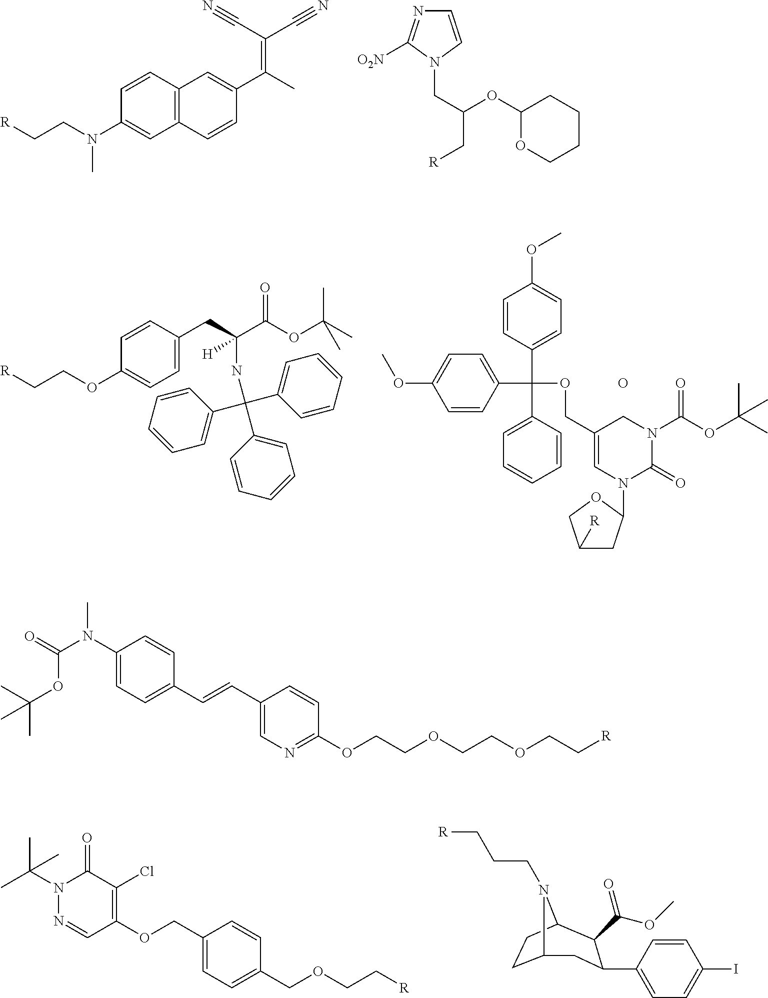
wherein R is

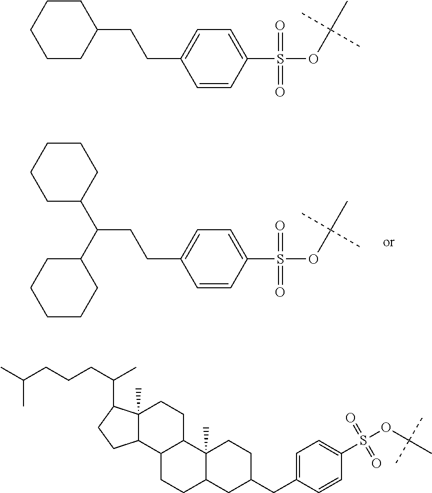

10. A modified leaving group L^{M} is selected from

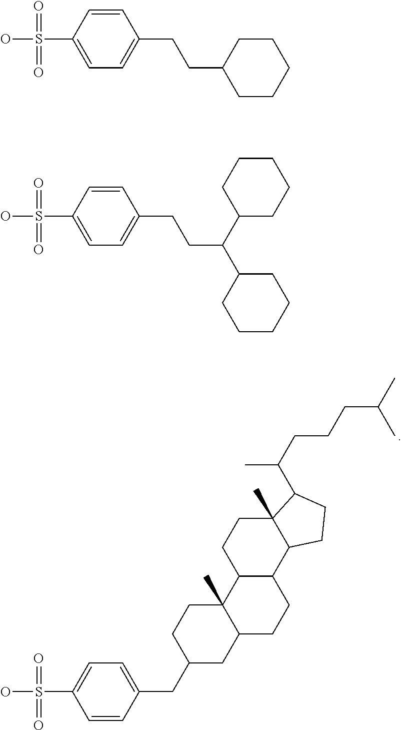

11. A method for obtaining compound of formula I according to claim 7 by reacting compound of formula III with a vector wherein
R1-L^{M1} (III)
R1 is halide and covalently bound to S* and
L^{M1} is [chemical structure]
